(19) United States
(12) Patent Application Publication
(10) Pub. No.: US 2023/0206159 A1
(43) Pub. Date: Jun. 29, 2023

(54) ANALYSIS SYSTEM, ANALYSIS DEVICE, ANALYSIS METHOD, PROGRAM, AND RECORDING MEDIUM

(71) Applicant: Murata Manufacturing Co., Ltd., Kyoto (JP)

(72) Inventors: Takumi ONO, Kyoto (JP); Toru YABE, Kyoto (JP); Koji TANAKA, Kyoto (JP)

(21) Appl. No.: 18/146,532

(22) Filed: Dec. 27, 2022

(30) Foreign Application Priority Data
Dec. 28, 2021 (JP) 2021-214656
Oct. 20, 2022 (JP) 2022-168351

(51) Int. Cl. G06Q 10/0639

(52) U.S. Cl. CPC G06Q 10/06393

(57) ABSTRACT

In an analysis system for analyzing a fatigue and/or stress state of an organization, the analysis system includes an information acquisition unit, an information storage unit, a fatigue and/or stress value acquisition unit, the fatigue and/or stress value being calculated based on the information regarding the fatigue and/or the stress, an adjustment target extraction unit configured to extract adjustment targets from among a plurality of members based on personal information including health information and a predetermined condition, the predetermined condition including whether a predetermined health state is good, and a fatigue and/or stress state analysis unit configured to adjust a set of the fatigue and/or stress values by correcting or excluding from an analysis the fatigue and/or stress value of the adjustment targets and to analyze the fatigue and/or stress state of the organization based on an adjusted set of the fatigue and/or stress values.

### CROSS REFERENCE TO RELATED APPLICATION

This application claims priority from Japanese Patent Application No. 2021-214656 filed on Dec. 28, 2021 and Japanese Patent Application No. 2022-168351 filed on Oct. 20, 2022. The contents of these applications are incorporated herein by reference in their entireties.

### BACKGROUND ART

The present disclosure relates to an analysis system, an analysis device, an analysis method, a program, and a recording medium each for use in analyzing a fatigue and/or stress state of an organization.

Japanese Unexamined Patent Application Publication No. 2000-311192 discloses a lifestyle improvement support system for determining a stress state of a person and giving an advice depending on the stress state.

The lifestyle improvement support system disclosed in Japanese Unexamined Patent Application Publication No. 2000-311192 still has room for improvement in the viewpoint of increasing the accuracy in analyzing a fatigue and/or stress state of an organization.

### BRIEF SUMMARY

The present disclosure provides an analysis system, an analysis device, an analysis method, a program, and a recording medium with each of which the accuracy in analyzing a fatigue and/or stress state of an organization is improved.

As aspect of the present disclosure provides an analysis system for analyzing a fatigue and/or stress state of an organization to which a plurality of members belongs, the analysis system including an information acquisition unit configured to acquire information regarding fatigue and/or stress of the plurality of members, an information storage unit configured to store personal information of the plurality of members, a fatigue and/or stress value acquisition unit configured to acquire a fatigue and/or stress value for each of the plurality of members, the fatigue and/or stress value being calculated based on the information regarding the fatigue and/or the stress, an adjustment target extraction unit configured to extract the member of an adjustment target from among the plurality of members based on the personal information including health information of the plurality of members and a predetermined condition for extracting the member of the adjustment target, the predetermined condition including whether a predetermined health state included in the health information is good, a fatigue and/or stress value adjustment unit configured to adjust a set of the fatigue and/or stress values by correcting the fatigue and/or stress value of the member of the adjustment target or by excluding the fatigue and/or stress value thereof from an analysis, and a fatigue and/or stress state analysis unit configured to analyze the fatigue and/or stress state of the organization based on an adjusted set of the fatigue and/or stress values.

As aspect of the present disclosure provides an analysis device for analyzing a fatigue and/or stress state of an organization to which a plurality of members belongs, the analysis device including a fatigue and/or stress value acquisition unit configured to acquire a fatigue and/or stress value for each of the plurality of members, the fatigue and/or stress value being calculated based on information regarding fatigue and/or stress of the plurality of members, an adjustment target extraction unit configured to acquire the member of an adjustment target from among the plurality of members, the member of the adjustment target being extracted based on personal information of the plurality of members, the personal information including health information of the plurality of members, and on a predetermined condition for extracting the member of the adjustment target, the predetermined condition including whether a predetermined health state included in the health information is good, a fatigue and/or stress value adjustment unit configured to acquire a set of the fatigue and/or stress values, the set of the fatigue and/or stress values being adjusted by correcting the fatigue and/or stress value of the member of the adjustment target or by excluding the fatigue and/or stress value thereof from an analysis, and a fatigue and/or stress state analysis unit configured to analyze the fatigue and/or stress state of the organization based on the adjusted set of the fatigue and/or stress values.

As aspect of the present disclosure provides an analysis method for analyzing a fatigue and/or stress state of an organization to which a plurality of members belongs, the analysis method including the steps of acquiring information regarding fatigue and/or stress of the plurality of members, acquiring personal information of the plurality of members, calculating a fatigue and/or stress value for each of the plurality of members based on the information regarding the fatigue and/or the stress, extracting the member of an adjustment target from among the plurality of members based on the personal information including health information of the plurality of members and a predetermined condition for extracting the member of the adjustment target, the predetermined condition including whether a predetermined health state included in the health information is good, adjusting a set of the fatigue and/or stress values by correcting the fatigue and/or stress value of the member of the adjustment target or by excluding the fatigue and/or stress value thereof from an analysis on condition that the personal information takes the predetermined value, and analyzing the fatigue and/or stress state of the organization based on an adjusted set of the fatigue and/or stress values.

As aspect of the present disclosure provides a program causing a computer to execute the above-described method.

As aspect of the present disclosure provides a recording medium on which the above-described program is recorded.

According to the present disclosure, the analysis system, the analysis device, the analysis method, the program, and the recording medium are provided with each of which the accuracy in analyzing the fatigue and/or stress state of the organization is improved.

### BRIEF DESCRIPTION OF THE DRAWINGS

FIG. 1 is a block diagram illustrating an analysis system according to Embodiment 1;

FIG. 2 is a table indicating fatigue and/or stress values of individual members belonging to an organization and personal information of the members;

FIG. 3 is a table indicating data of a heartbeat interval of one member;

FIG. 4 is a table indicating an analysis result of a fatigue and/or stress state of the organization, the result being obtained with a fatigue and/or stress state analysis unit;

FIG. 5 is a flowchart referred to for explanation of an analysis method in the analysis system of FIG. 1;

FIG. 6 is a flowchart referred to for explanation of steps of extracting a member of an adjustment target:

FIG. 7 is a table indicating an analysis result of the fatigue and/or stress state of the organization according to Modification 1 of Embodiment 1;

FIG. 8 is a block diagram illustrating an analysis system according to Modification 2 of Embodiment 1;

FIG. 9 is a chart representing an example of an analysis result displayed on a display unit in the analysis system of FIG. 8;

FIG. 10 is a chart representing another example of the analysis result displayed on the display unit in the analysis system of FIG. 8;

FIG. 11 is a flowchart illustrating steps of extracting a member of the adjustment target in an analysis system according to Embodiment 2:

FIG. 12 is a table indicating the fatigue and/or stress values of the individual members belonging to the organization and personal information of the members;

FIG. 13 is a table indicating correction coefficients for different health states;

FIG. 14 is a graph indicating an example of statistical data used in the analysis system according to Modification 1 of Embodiment 2;

FIG. 15 is a flowchart illustrating steps of extracting a member of the adjustment target in an analysis system according to Embodiment 3:

FIG. 16 is a table indicating the fatigue and/or stress values of the individual members belonging to the organization and personal information of the members;

FIG. 17 is a block diagram illustrating an analysis system according to Embodiment 4;

FIG. 18 is a flowchart illustrating an analysis method in the analysis system 2 of FIG. 17; and

FIG. 19 is a block diagram illustrating a configuration of an analysis system 3 according to Embodiment 5.

### DETAILED DESCRIPTION

### Underlying Knowledge for Present Disclosure

Japanese Unexamined Patent Application Publication No. 2000-311192 discloses the lifestyle improvement support system for determining a stress state of a person and giving an advice depending on the stress state. In the lifestyle improvement support system disclosed in Japanese Unexamined Patent Application Publication No. 2000-311192, the stress state is determined based on, for example, data regarding living conditions of a person, biological data such as weight and body temperature of the person, or the like.

Recently, there has been a demand for utilizing information regarding fatigue and/or stress for each of persons belonging to an organization in operations of the organization. For instance, it has been studied to improve work efficiency by analyzing a fatigue and/or stress state of an organization and by, if there is any member whose fatigue and/or stress state is deteriorated due to work and other factors related to the organization, taking action to reduce, for example, a workload of the member in question. Here, the wording “fatigue and/or stress state of an organization” indicates, for example, what percentage of the members belonging to the organization are in a highly fatigued and/or stressed state, and it includes statistics of fatigue and/or stress values of the individual members. The wording “highly fatigued and/or stressed state” indicates a state in which at least one of fatigue or stress is high.

In the case of analyzing the fatigue and/or stress state based on the data regarding a person or the biological data of the person as in Japanese Unexamined Patent Application Publication No. 2000-311192, for example, deterioration of the stress state caused by factors not related to the organization may become noise in analysis of the stress state of the organization. The wording “deterioration of the fatigue and/or stress state caused by factors not related to the organization” indicates, for example, deterioration of the fatigue and/or stress state attributable to a health state of the member or life events, such as ceremonial occasions, occurred on the member.

The inventor(s) have achieved the present disclosure, described below, as a result of finding that the accuracy in analyzing the fatigue and/or stress state of the organization can be improved by regarding the above-mentioned deterioration of the fatigue and/or stress state due to the personal matters as the noise in the analysis and by reducing that noise.

Embodiments of the present disclosure will be described below with reference to the accompanying drawings.

### Embodiment 1

### Overall Configuration

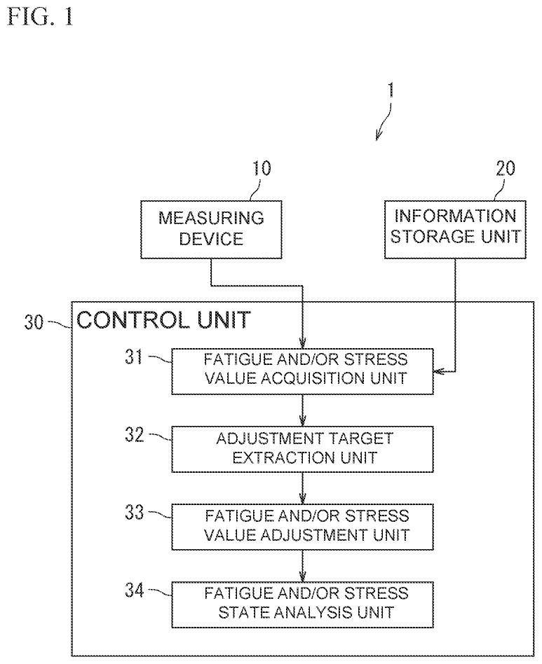

FIG. 1 is a block diagram illustrating an analysis system according to Embodiment 1. The analysis system 1 is described with reference to FIG. 1.

The analysis system 1 is a system for analyzing a fatigue and/or stress state of an organization to which a plurality of members belongs. The analysis system 1 analyzes the fatigue and/or stress state of the organization based on information regarding fatigue and/or stress of the plurality of members. The analysis system 1 includes a measuring device 10, an information storage unit 20, and a control unit 30. The information regarding the fatigue and/or the stress just needs to include at least one of information regarding the fatigue or information regarding the stress and is not always required to include the information regarding both of the fatigue and the stress. The fatigue and/or stress state of the organization indicates, for example, a percentage of members who have a high fatigue and/or stress level among the members belonging to the organization, the presence of one or more members with a high fatigue level, or the presence of one or more members with a high stress level, and it includes statistics of the fatigue and/or stress values of the individual members.

### Measuring Device

The measuring device 10 is a device for acquiring the information regarding the fatigue and/or the stress of the plurality of members. The information regarding the fatigue and/or the stress includes information, such as biological information, a voice tone, or a body motion, for each of the plurality of members, the information enabling at least one of a fatigue level or a stress level of each member to be measured or estimated. The fatigue level represents how fatigued the member is, and the stress level represents how stressed the member is. Both or either one of the fatigue level and the stress level is referred to together as a fatigue and/or stress level in some cases.

In this embodiment, the measuring device 10 is constituted by, for example, a biological sensor capable of measuring the biological information of the member. The biological information is given as various items of physiological and anatomical information obtained from a living body. The biological information includes information, such as a body temperature, a heartbeat, a pulsation, a respiratory rate, and an electroencephalogram, for example.

In this embodiment, the measuring device 10 is a device capable of measuring a heartbeat interval as the biological information of the member. Alternatively, the measuring device 10 may measure the pulsation as the biological information of the member. The term “heartbeat interval” indicates an interval between the occurrence time of one R wave and the occurrence time of a next R wave in an electrocardiogram. Usually, a periodical fluctuation is observed in the heartbeat interval. The periodical fluctuation in the heartbeat interval is called a heartbeat fluctuation. An index indicating the fatigue or stress state can be calculated by using the heartbeat fluctuation. The index indicating the fatigue or stress state is, for example, an autonomic nerve index. Alternatively, an active oxygen concentration in blood, a value of lipid contained in blood, total protein in blood, or a concentration of amino acid in blood can also be used as the index indicating the fatigue or stress state. In addition, the index indicating the fatigue or stress state can be further calculated from the result of a test on, for example, activity of HHV6 (human herpes virus) in salvia, a PVT test for measuring a fatigue level from a reaction speed, or a test with an activity meter using an acceleration or GPS. In this embodiment, the index indicating at least one of the fatigue level or the stress level is referred to as the “fatigue and/or stress value”.

The measuring device 10 is a device for measuring the heartbeat interval, such as a heartbeat sensor, for example. Alternatively, the measuring device 10 may be a wearable device such as a smart watch including a heartbeat sensor.

The information regarding the fatigue and/or the stress, acquired by the measuring device 10, is stored into, for example, a storage unit in the control unit 30 described later.

### Information Storage Unit

The information storage unit 20 stores personal information of the plurality of members. The personal information includes, for example, health information, information regarding the occurrence of the life events and so on, or information regarding work in the organization for each of the members.

The health information includes information regarding the health state of the member, such as results of medical examination, pre-existing diseases, history of current illness (presence of disease), the presence of allergy, and so on. The health information may further include a level of the health state. The level of the health state includes, for example, the severity of the disease, allergy, or the like. The information regarding the occurrence of the life events includes, for example, ceremonial occasions or situations of nursing or childcare, and is information possibly affecting the fatigue and/or stress level among various items of the personal information of the member. In more detail, the life events on the member oneself are, for example, start or end of school attendance, a job or a side job, marriage of the member oneself, including preparations for a wedding ceremony, divorce including preparations, such as divorce mediations, separation from a spouse, a disease, an accident, or the like. The life events on relatives or friends include, for example, pregnancy, childbirth, childcare, nursing, marriage, divorce, school attendance, transfer school, leaving school, start of a job, job change, retirement, or the like. The life events may further include moving, fare well to a relative or a friend due to, for example, an incident or an accident, or the like. Other examples of the life events include, for example, the case in which the member oneself or a relative become a victim of fraud, or in which the member oneself or a relative commits an incident or an accident or is involved therein. The information regarding the work in the organization includes, for example, overtime hours, frequency of business trips, transfer due to personnel change, and is information possibly affecting the fatigue and/or stress level among various items of the information related to the work of the member.

The information storage unit 20 is constituted by, for example, a memory or a storage device such as HDD (Hard Disk Drive) or SSD (Solid State Drive).

The personal information is entered through, for example, an input terminal of an electronic device or the like, such as a PC, a tablet PC, or a smart phone. The personal information may be entered, for example, in bulk by a representative member of the organization. Alternatively, each member may enter the personal information of oneself.

### Control Unit

The control unit 30 analyzes the fatigue and/or stress state of the organization based on the information regarding the fatigue and/or the stress and the personal information. The fatigue and/or stress state of the organization includes, for example, the statistics of the fatigue and/or stress values of the individual members.

The fatigue and/or stress state of the organization can be expressed by indicating, based on the fatigue and/or stress values of the members belonging to the organization, for example, the number or the percentage of the members with high fatigue and/or stress values, namely of the members with at least one of a high fatigue level or a high stress level, among all the members belonging to the organization.

In this embodiment, the control unit 30 includes a fatigue and/or stress value acquisition unit 31, an adjustment target extraction unit 32, a fatigue and/or stress value adjustment unit 33, and a fatigue and/or stress state analysis unit 34. Note that the control unit 30 corresponds to an “analysis device” in this embodiment.

The control unit 30 includes a universal processor, such as CPU or MPU, that realizes predetermined functions by executing programs. The control unit 30 further includes a storage unit (not illustrated). The control unit 30 realizes functions of the fatigue and/or stress value acquisition unit 31, the adjustment target extraction unit 32, the fatigue and/or stress value adjustment unit 33, and the fatigue and/or stress state analysis unit 34 by calling and executing control programs stored in the storage unit, for example. The control unit 30 is not limited to a unit for realizing the predetermined functions with cooperation of hardware and software and may be a hardware circuit that is designed with the dedicated purpose of realizing the predetermined functions. Thus, the control unit 30 can be realized with any of various processors, such as CPU, MPU, CPU, FPGA, DSP, and ASIC.

Individual components of the control unit 30 will be described below.

Fatigue and/or Stress Value Acquisition Unit

The fatigue and/or stress value acquisition unit acquires the fatigue and/or stress value for each of the plurality of members by calculating that value based on the information regarding the fatigue and/or the stress. The fatigue and/or stress value is calculated based on, for example, the heartbeat interval acquired by the measuring device 10.

In more detail, the autonomic nerve index is calculated by using LF (Low Frequency), namely a power spectrum in a low frequency band of the heartbeat fluctuation that is the periodical fluctuation in the heartbeat interval, and HF (High Frequency), namely a power spectrum in a high frequency band of the heartbeat fluctuation. LF mainly includes a sympathetic nerve component, and HF mainly includes a parasympathetic nerve component.

For instance, Total Power (TP) expressed by a total of LF and HF is the autonomic nerve index mainly related to the fatigue level and exhibits such a tendency that, as TP takes a smaller value, the fatigue level is higher. Instead, ccvTP obtained by correcting TP with a heart rate or a value (D-Score) representing ccvTP as a deviation value per age may be used as the index related to the fatigue level.

Furthermore, LF/HF indicating a balance between LF and HF, namely a balance between the sympathetic nerve and the parasympathetic nerve, represents activity of the sympathetic nerve and is an autonomic nerve index related to the stress level. LF/HF exhibits such a tendency that, as it takes a greater value, the stress level is higher.

In this embodiment, the fatigue and/or stress value acquisition unit 31 calculates the autonomic nerve index such as TP or LF/HF, for example, as the fatigue and/or stress value. For instance, the fatigue and/or stress value acquisition unit 31 may calculate either one of TP indicating the fatigue level or LF/HF indicating the stress level, or may calculate both of TP and LF/HF.

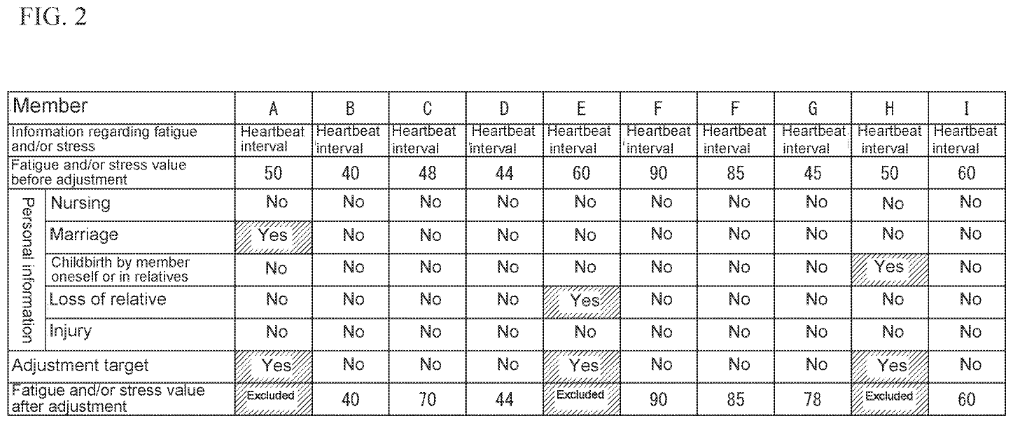

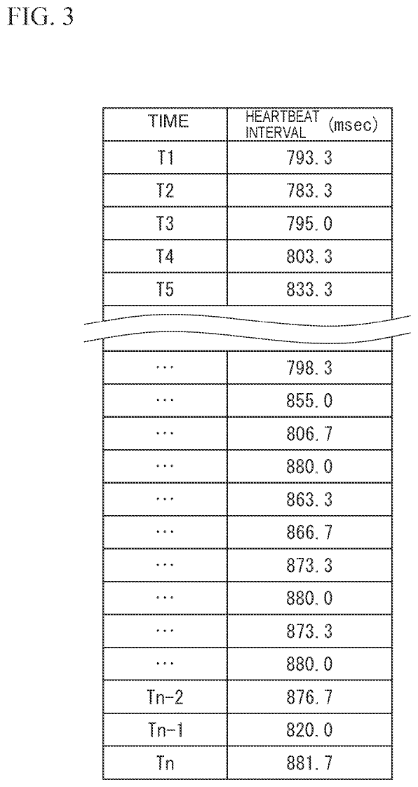

FIG. 2 is a table indicating the fatigue and/or stress values of members A to I belonging to an organization and personal information of the members. FIG. 3 is a table indicating data of the heartbeat interval of one member.

As illustrated in FIG. 2, in this embodiment, the heartbeat interval for each of the members A to I is acquired as the information regarding the fatigue and/or the stress. As illustrated in FIG. 3, by way of example, the heartbeat interval is acquired for a predetermined period (from time T1 to time Tn) on a time serial basis. The fatigue and/or stress value is calculated by the fatigue and/or stress value acquisition unit 31 based on the heartbeat interval illustrated in FIG. 3. In this embodiment, the autonomic nerve index is calculated based on the heartbeat interval. Note that the fatigue and/or stress value denoted in FIG. 2 represents TP.

The fatigue and/or stress value (“Fatigue and/or stress value before adjustment” in FIG. 2) calculated by the fatigue and/or stress value acquisition unit 31 is stored into, for example, the storage unit in the control unit 30.

### Adjustment Target Extraction Unit

The adjustment target extraction unit 32 extracts a member of an adjustment target from among the plurality of members belonging to the organization based on the personal information stored in the information storage unit 20 and a predetermined condition for extracting the member of the adjustment target.

Among the members belonging to the organization, there may be a member whose fatigue and/or stress level is high because of a personal factor other than the factors related to the work in the organization attributable to, for example, the health state or the occurrence of a predetermined life event. The fatigue and/or stress value of that member is extracted by the adjustment target extraction unit 32 to exclude the member in question from an analysis on the fatigue and/or stress state of the organization. An analysis on the fatigue and/or the stress mainly caused by the factors related to the organization can be made after excluding, from the analysis, the fatigue and/or stress value of the member whose fatigue and/or stress level is high because of the personal factor.

As illustrated in FIG. 2, the personal information includes the health information of the member or the life event occurred on the member. In this embodiment, the occurrence or not of the life event, such as nursing, marriage, childbirth, loss, or the like, and the presence of an injury caused by an accident are used as the personal information. The life event is not limited to the above-mentioned examples, and other various life events, such as childcare, moving, and divorce, can also be used as the personal information. In addition, the health state of the member, including the presence of disease, results of medical examination, and so on, can be further used as the health information.

The predetermined condition includes whether a predetermined health state included in the health information is good, or whether the predetermined life event has occurred.

When it is found, by referring to the personal information stored in the information storage unit, that the predetermined health state of any member is not good or the predetermined life event has occurred on any member, the adjustment target extraction unit 32 extracts the member in question as the member of the adjustment target.

For instance, in the example of FIG. 2, the member A is extracted as the member of the adjustment target due to marriage as the occurrence of the life event. Similarly, the member E is extracted as the member of the adjustment target due the loss of a relative as the occurrence of the life event. Moreover, the member H is extracted as the member of the adjustment target due to childbirth by the member oneself or in relatives as the occurrence of the life event.

The member extracted as the adjustment target by the adjustment target extraction unit 32 is stored into, for example, the storage unit in the control unit 30.

Fatigue and/or Stress Value Adjustment Unit

The fatigue and/or stress value adjustment unit 33 adjusts a set of the fatigue and/or stress values by correcting the fatigue and/or stress value of the member of the adjustment target or by excluding the fatigue and/or stress value thereof from the analysis. In this embodiment, the fatigue and/or stress value adjustment unit 33 adjusts a set of the fatigue and/or stress values by excluding the fatigue and/or stress value of the member of the adjustment target from an analysis target.

The wording “set of the fatigue and/or stress values” indicates a set of the fatigue and/or stress values of the individual members A to I illustrated in FIG. 2. The fatigue and/or stress value adjustment unit 33 collects and stores the fatigue and/or stress values of the individual members A to I into the storage unit in the control unit 30, thus forming the set of the fatigue and/or stress values. The fatigue and/or stress value adjustment unit 33 adjusts the set of the fatigue and/or stress values by excluding the fatigue and/or stress values of the members A, E and H who have been extracted as the adjustment targets. As a result, the adjusted set of the fatigue and/or stress values is constituted by the fatigue and/or stress values after excluding the fatigue and/or stress values of the individual members A, E and H, namely by the fatigue and/or stress values of the members B to D, F, G and I.

The set of the fatigue and/or stress values after having been adjusted by the fatigue and/or stress value adjustment unit 33 is stored into, for example, the storage unit in the control unit 30.

Fatigue and/or Stress State Analysis Unit

The fatigue and/or stress state analysis unit 34 analyzes the fatigue and/or stress state of the organization based on the set of the fatigue and/or stress values after having been adjusted by the fatigue and/or stress value adjustment unit 33.

The analysis on the stress state of the organization includes, for example, classification of the adjusted set of the fatigue and/or stress values into a plurality of categories depending on the magnitudes of the fatigue and/or stress values. The fatigue and/or stress values in FIG. 2 each represent TP, and a lower value of TP indicates a higher fatigue and/or stress level. The fatigue and/or stress state analysis unit 34 classifies the fatigue and/or stress values into three categories of “Low”, “Medium”, and “High” depending on the magnitudes of those values. In other words, the fatigue and/or stress state analysis unit 34 classifies the value representing a high fatigue and/or stress level into the category of “High”, the value representing a medium fatigue and/or stress level into the category of “Medium”, and the value representing a low fatigue and/or stress level into the category of “Low”.

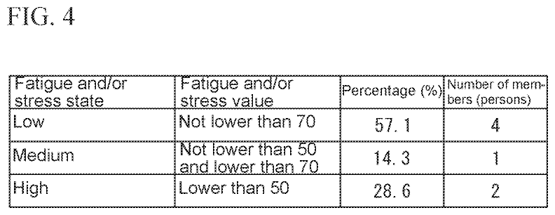

The classification of the fatigue and/or stress values may be performed based on, for example, predetermined thresholds. For instance, the fatigue and/or stress values may be classified depending on the magnitudes of the fatigue and/or stress values such that the value lower than a first threshold is classified into the category of “High”, the value not lower than the first threshold and lower than a second threshold is classified into the category of “Medium”, and the value not lower than the second threshold is classified into the category of “Low”. In the example of FIG. 2, the first threshold is 50, and the second threshold is 70 (see FIG. 4).

FIG. 4 is a table indicating an analysis result of the fatigue and/or stress state of the organization, the analysis result being obtained with the fatigue and/or stress state analysis unit 34. As illustrated in FIG. 4, in the organization to which the members A to I belong, the fatigue and/or stress values of the individual members are classified into the three categories by the fatigue and/or stress state analysis unit 34. The classification provides the analysis result that, among seven members after excluding the members A, E and H as the adjustment targets, the members of 57.1% have the low fatigue and/or stress level, the members of 14.3% have the medium fatigue and/or stress level, and the members of 28.6% have the high fatigue and/or stress level.

### Analysis Method

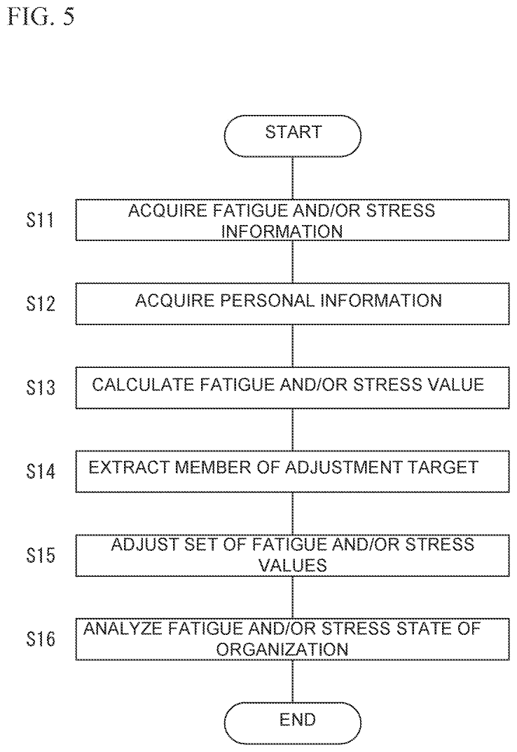

FIG. 5 is a flowchart referred to for explanation of an analysis method in the analysis system 1 of FIG. 1. The analysis method in the analysis system 1 is described with reference to FIG. 5.

First, the control unit 30 acquires the information regarding the fatigue and/or the stress of the plurality of individual members (step S11). The information regarding the fatigue and/or the stress is, for example, information of the heartbeat interval for each member measured by the measuring device 10 capable of acquiring the heartbeat interval and is stored into, for example, the storage unit in the control unit 30.

Then, the control unit 30 acquires the personal information of the plurality of members (step S12). The personal information is entered through, for example, an input terminal, such as a PC, a tablet PC, or a smart phone by each member or a representative member of the organization and is stored into the information storage unit 20. The control unit 30 acquires the personal information by referring to the personal information stored in the information storage unit 20 or by loading the personal information into the storage unit in the control unit 30.

Then, the fatigue and/or stress value acquisition unit 31 calculates the fatigue and/or stress value for each of the plurality of members based on the information regarding the fatigue and/or the stress acquired in the step S11 (step S13). As described above, the fatigue and/or stress value is the autonomic nerve index calculated based on the heartbeat interval, for example, and TP is calculated in this embodiment.

Then, the adjustment target extraction unit 32 extracts the member of the adjustment target from among the plurality of members based on the personal information and the predetermined condition for extracting the member of the adjustment target (step S14). The wording “the member of the adjustment target” indicates the member for whom the magnitude of the fatigue and/or stress value is to be corrected or who is to be excluded from the target of the analysis on the fatigue and/or stress state of the organization.

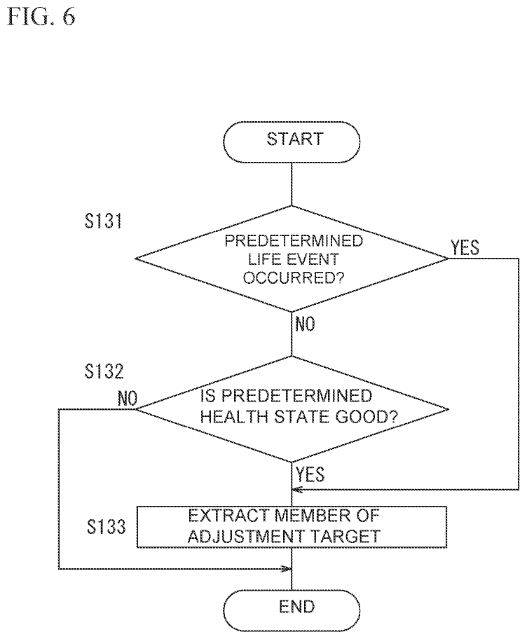

FIG. 6 is a flowchart referred to for explanation of steps of extracting the member of the adjustment target. Details of the steps of extracting the member of the adjustment target is described with reference to FIG. 6.

The adjustment target extraction unit 32 determines whether the predetermined life event has occurred on the member (step S131). If the occurrence of the predetermined life event is included in the personal information of the member which has been referred to by the adjustment target extraction unit 32 (Yes in step S131), the adjustment target extraction unit 32 advances to a step S133. As illustrated in the example of FIG. 2, the predetermined life event includes, for example, nursing, marriage, childbirth, loss, and so on. If the occurrence of the predetermined life event is not included in the personal information of the member which has been referred to by the adjustment target extraction unit 32 (No in step S131), the adjustment target extraction unit 32 advances to a step S132.

If the predetermined life event has not occurred (No in step S131), the adjustment target extraction unit 32 determines whether the predetermined health state included in the health information of the member is good (step S132). If the personal information of the member indicates, for example, that the member has a disease or an injury, the adjustment target extraction unit 32 determines that the health state is not good (No in step S132) and advances to the step S133. If the personal information of the member does not indicate that the member has a disease or an injury (Yes in step S131), the adjustment target extraction unit 32 ends the steps of extracting the employee of the adjustment target.

If the predetermined life event has occurred on the member (Yes in step S131) or if the health state is not good, the member in question is extracted as the adjustment target. For instance, in the example of FIG. 2, the member A is extracted as the adjustment target because of the occurrence of the predetermined life event (marriage). Likewise, the members E and H are also extracted as the adjustment targets.

Thus, in the steps of extracting the member of the adjustment target, the member to be extracted as the adjustment target is decided by, for each of the members, determining whether the predetermined life event has occurred and further determining whether the health state is good.

Returning to FIG. 5, after extracting the member of the adjustment target, the fatigue and/or stress value adjustment unit 33 adjusts the fatigue and/or stress value of the member of the adjustment target (step S15). In this embodiment, the fatigue and/or stress value adjustment unit 33 excludes the fatigue and/or stress value of the member of the adjustment target from the analysis target. Hence, as illustrated in FIG. 2, the set of the fatigue and/or stress values is adjusted to a set of the fatigue and/or stress values of the seven members among the ten members A to I belonging to the organization except for the fatigue and/or stress values of the members A, E and H.

Then, the fatigue and/or stress state analysis unit 34 analyzes the fatigue and/or stress state of the organization (step S16). The fatigue and/or stress state of the organization is analyzed based on the set of the fatigue and/or stress values after having been adjusted in the step S15. For instance, in the example of FIG. 2, the adjusted set of the fatigue and/or stress values is classified into the three categories with the fatigue and/or stress values of lower than 50, not lower than 50 and lower than 70, and not lower than 70. The analysis result illustrated in FIG. 4 can be obtained by calculating, for example, the percentage or the number of the members belonging to each category with respect to the entire set of the fatigue and/or stress values.

### Effects

According to Embodiment 1, the following effects can be obtained.

The analysis system 1 is an analysis system for analyzing a fatigue and/or stress state of an organization to which a plurality of members belongs and includes the measuring device 10, the information storage unit 20, and the control unit 30. The measuring device 10 acquires the information regarding the fatigue and/or the stress of the plurality of members. The information storage unit 20 stores the personal information of the plurality of members. The control unit 30 analyzes the fatigue and/or stress state of the organization based on the information regarding the fatigue and/or the stress and the personal information. The fatigue and/or stress value acquisition unit 31 in the control unit 30 calculates the fatigue and/or stress value for each of the plurality of members based on the information regarding the fatigue and/or the stress. The adjustment target extraction unit 32 in the control unit 30 extracts the member of the adjustment target from among the plurality of members based on the personal information and the predetermined condition for extracting the member of the adjustment target. The fatigue and/or stress value adjustment unit 33 in the control unit 30 adjusts the set of the fatigue and/or stress values by correcting the fatigue and/or stress value of the member of the adjustment target or by excluding the fatigue and/or stress value thereof from the analysis. The fatigue and/or stress state analysis unit 34 in the control unit 30 analyzes the fatigue and/or stress state of the organization based on the adjusted set of the fatigue and/or stress values.

With the configuration described above, the analysis system can be provided which has succeeded in improving the accuracy in analyzing the fatigue and/or stress state of the organization. In more detail, the fatigue and/or stress state of the organization depending on the factors related to the organization can be analyzed by extruding, from the analysis target, the fatigue and/or stress value of the member with the high fatigue or stress level due to the factors not related to the organization. Since the factors not related to the organization are excluded, the analysis result of the fatigue and/or stress state can be usefully applied to improve operations of the organization.

For instance, when the life event, such as marriage, childbirth, loss, or the like, has occurred on any member, or when the health state of any member is not good, that member may temporarily come into a state with the high fatigue and/or stress level. If the fatigue and/or stress value of the member in question is included in the analysis on the fatigue and/or stress state of the organization, this may lead to an analysis result that the fatigue and/or stress state is deteriorated although the operations of the organization are appropriate. Thus, the fatigue and/or stress state of the organization can be accurately analyzed by extruding, from the analysis target, the fatigue and/or stress value of the member for whom the fatigue and/or stress level is estimated to be temporality high due to the occurrence of the life event or the health state being not good.

The personal information includes the health information of the plurality of members or the occurrence of the life events on the plurality of members. The predetermined condition includes whether the predetermined health state included in the health information is good, or whether the predetermined life event has occurred. If the predetermined health state is not good, or if the predetermined life event has occurred, the adjustment target extraction unit 32 in the control unit 30 extracts the member in question as the adjustment target. The fatigue and/or stress value adjustment unit 33 in the control unit 30 adjusts the set of the fatigue and/or stress values by excluding the fatigue and/or stress value of the member of the adjustment target from the analysis.

With the configuration described above, the fatigue and/or stress value of the member for whom the fatigue and/or stress level is estimated to be temporality high can be excluded from the analysis target, and the accuracy in analyzing the fatigue and/or stress state of the organization can be improved.

The fatigue and/or stress state analysis unit 34 in the control unit 30 analyzes the fatigue and/or stress state of the organization by classifying the fatigue and/or stress values into the plurality of categories depending on the magnitudes of the fatigue and/or stress values.

With the configuration described above, the fatigue and/or stress state in the entirety of the organization instead of the individual members can be easily grasped.

The information regarding the fatigue and/or the stress includes biological information of the plurality of members.

With the configuration described above, the fatigue and/or stress state can be analyzed by using information that is obtained from the biological information, such as the autonomic nerve index, for example.

The analysis device includes the fatigue and/or stress value acquisition unit 31, the adjustment target extraction unit 32, the fatigue and/or stress value adjustment unit 33, and the fatigue and/or stress state analysis unit 34. The fatigue and/or stress value acquisition unit 31 calculates the fatigue and/or stress value for each of the plurality of members based on the information regarding the fatigue and/or the stress of the plurality of members. The adjustment target extraction unit 32 extracts the member of the adjustment target from among the plurality of members based on the personal information of the plurality of members and the predetermined condition for extracting the member of the adjustment target. The fatigue and/or stress value adjustment unit 33 adjusts the set of the fatigue and/or stress values by correcting the fatigue and/or stress value of the member of the adjustment target or by excluding the fatigue and/or stress value thereof from the analysis target. The fatigue and/or stress state analysis unit 34 analyzes the fatigue and/or stress state of the organization based on the adjusted set of the fatigue and/or stress values.

With the configuration described above, the analysis device can be provided which has succeeded in improving the accuracy in analyzing the fatigue and/or stress state of the organization.

The analysis method includes the step S11 of acquiring the information regarding the fatigue and/or the stress, the step S12 of acquiring the personal information, the step S13 of calculating the fatigue and/or stress value, the step S14 of extracting the member of the adjustment target, the step S15 of adjusting the set of the fatigue and/or stress values, and the step S16 of analyzing the fatigue and/or stress state of the organization.

With the feature described above, the analysis method can be provided which has succeeded in improving the accuracy in analyzing the fatigue and/or stress state of the organization.

While the above embodiment has been described in connection with the example in which the heartbeat interval is used as the information regarding the fatigue and/or the stress, the present disclosure is not limited to the above-mentioned example. The information regarding the fatigue and/or the stress may be another type of biological information, such as a body temperature, a heartbeat, a pulsation, a respiratory rate, or an electroencephalogram, for example. Other types of information, including a voice tone or a body motion of the member, and so on, than the biological information can also be used as the information regarding the fatigue and/or the stress.

The predetermined life event may include an event occurred during a period from a past predetermined time to an acquisition time of the information regarding the fatigue and/or the stress. In other words, information of a life event having occurred during a predetermined period, for example, a period from the acquisition time of the information regarding the fatigue and/or the stress to one year ago, may be used to extract the member of the adjustment target. Because change in the fatigue and/or stress level due to the occurrence of the life event is temporary in many cases, the fatigue and/or stress state of the organization can be analyzed with high accuracy by excluding the life events that have occurred outside of the predetermined period.

While the above embodiment has been described in connection with the example in which the analysis system 1 acquires the information regarding the fatigue and/or the stress by measuring that information with the measuring device 10, the present disclosure is not limited to the above-mentioned example. In another example, the analysis system 1 may acquire the information regarding the fatigue and/or the stress through communication via a network by using a communication circuit. Thus, in the analysis system 1, the configuration for acquiring the information regarding the fatigue and/or the stress is not limited to the measuring device 10 and just needs to be realized with an information acquisition unit that is configured to acquire the information regarding the fatigue and/or the stress.

While the above embodiment has been described in connection with the example in which the fatigue and/or stress value acquisition unit 31 acquires the fatigue and/or stress value for each of the plurality of members by calculating that value based on the information regarding the fatigue and/or the stress, the present disclosure is not limited to the above-mentioned example. The fatigue and/or stress value acquisition unit 31 just needs to be able to acquire the fatigue and/or stress value for each of the plurality of members, that value being calculated based on the information regarding the fatigue and/or the stress of the plurality of members. In another example, the information regarding the fatigue and/or the stress acquired by the measuring device 10 may be sent to another device (external device) different from the analysis device (the control unit 30), and the fatigue and/or stress value may be calculated in the different device. For instance, the fatigue and/or stress value acquisition unit 31 may be constituted by a communication circuit communicating with the different device via a network. In such a case, the fatigue and/or stress value acquisition unit 31 can acquire information about the information of the fatigue and/or the stress, calculated in the different device, through communication with the different device via the network.

While the above embodiment has been described in connection with the example in which the adjustment target extraction unit 32 acquires the member of the adjustment target from among the plurality of members belonging to the organization by extracting that member based on the personal information and the predetermined condition for extracting the member of the adjustment target, both being stored in the information storage unit 20, the present disclosure is not limited to the above-mentioned example. The adjustment target extraction unit 32 just needs to be able to acquire the member of the adjustment target from among the plurality of members, that member being extracted based on the personal information of the plurality of members, including the health information of the plurality of members, and on the predetermined condition for extracting the member of the adjustment target, including whether the predetermined health state included in the health information is good. In another example, the personal information stored in the information storage unit 20 may be sent to another device (external device) different from the analysis device (the control unit 30), and the employee of the adjustment target may be extracted in the different device. The predetermined condition for extracting the member of the adjustment target may be previously stored in, for example, a storage in the different device or may be sent to the different device from the information storage unit 20. The different device acquires the personal information and the predetermined condition for extracting the member of the adjustment target, both being stored in the information storage unit 20, via a network and extracts the member of the adjustment target. The adjustment target extraction unit 32 may be constituted by, for example, a communication circuit communicating with the different device via the network. In such a case, the adjustment target extraction unit 32 can acquire information about the employee of the adjustment target, the information having been extracted by the different device, through communication with the different device via the network.

While the above embodiment has been described in connection with the example in which the fatigue and/or stress value adjustment unit 33 acquires the set of the fatigue and/or stress values through adjustment made by correcting the fatigue and/or stress value of the member of the adjustment target or by excluding the fatigue and/or stress value thereof from the analysis, the present disclosure is not limited to the above-mentioned example. The fatigue and/or stress value adjustment unit 33 just needs to be able to acquire the set of the fatigue and/or stress values, that set being adjusted by correcting the fatigue and/or stress value of the member of the adjustment target or by excluding the fatigue and/or stress value thereof from the analysis. In another example, the information regarding the fatigue and/or the stress acquired by the measuring device 10 and information regarding the employee of the adjustment target extracted by the adjustment target extraction unit 32 may be sent to another device (external device) different from the analysis device (the control unit 30), and the set of the fatigue and/or stress values may be adjusted in the different device. For instance, the fatigue and/or stress value adjustment unit 33 may be constituted by a communication circuit communicating with the different device via a network. In such a case, the fatigue and/or stress value adjustment unit 33 can acquire information about the set of the fatigue and/or stress values after having been adjusted by the different device through communication with the different device via the network.

While the above embodiment has been described in connection with the example in which the fatigue and/or stress state analysis unit 34 analyzes the fatigue and/or stress state of the organization based on the adjusted set of the fatigue and/or stress values, the present disclosure is not limited to the above-mentioned example. The fatigue and/or stress state analysis unit 34 is not always required to perform the analysis only in the analysis device (the control unit 30). Data for the analysis may be sent to a different device (external device), and part of analytical processing may be performed in the different device. In such a case, the analysis device can acquire part of the analysis result through communication with the different device via a network and can reduce a time taken for the analytical processing and a load exerted on the analysis device.

While the above embodiment has been described in connection with the example in which the information storage unit 20 is a memory or a storage device such as HDD, the present disclosure is not limited to the above-mentioned example. The information storage unit 20 may be constituted by, for example, a communication circuit, and information stored in a server different from the analysis system 1 may be acquired via a network.

The above-described embodiment can be applied to a program as well. Moreover, the above-described embodiment can be recorded as a program on a recording medium.

### Modifications

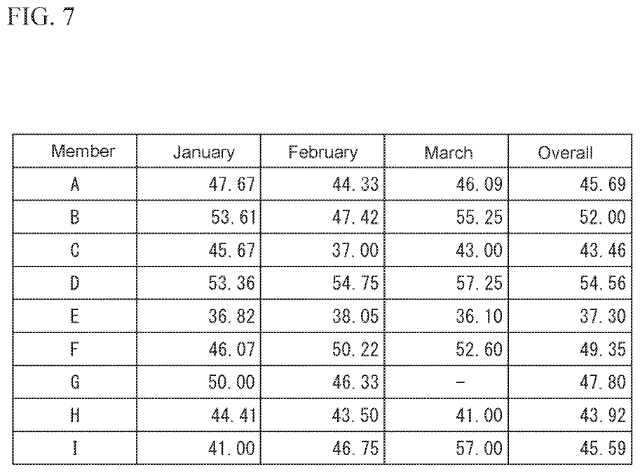

FIG. 7 is a table indicating an analysis result of the fatigue and/or stress state of the organization according to Modification 1 of Embodiment 1. As illustrated in FIG. 7, the fatigue and/or stress state of the organization may be represented by classifying the set of the fatigue and/or stress values per month, for example. Classifying the set of the fatigue and/or stress values per month makes it possible to grasp the magnitude of the fatigue and/or stress value of each member per month or to find the member whose fatigue and/or stress level tends to rise.

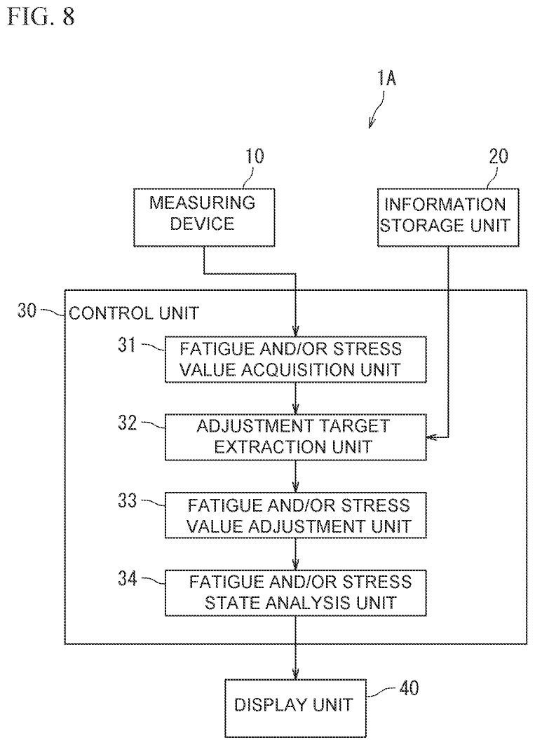

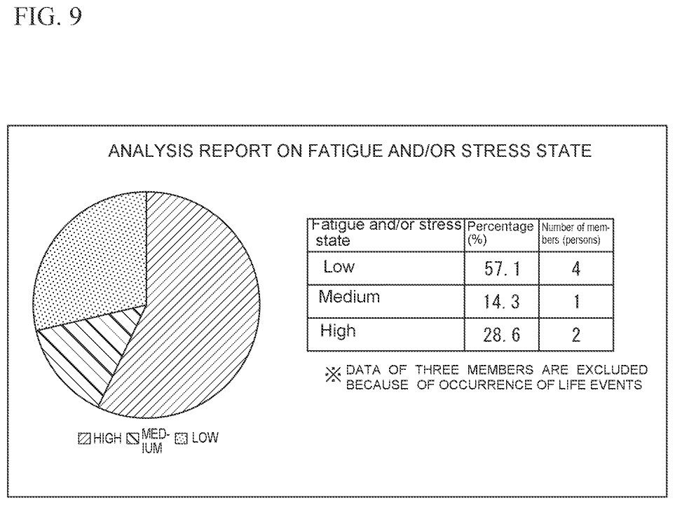

FIG. 8 is a block diagram illustrating an analysis system 1A according to Modification 2 of Embodiment 1. FIG. 9 is a chart representing an example of an analysis result displayed on a display unit 40 of the analysis system 1A of FIG. 8.

The analysis system 1A according to Modification 2 of Embodiment 1 includes the display unit 40 for displaying the analysis result of the fatigue and/or stress state of the organization, and the analysis result of the fatigue and/or stress state of the organization is graphically displayed on the display unit 40.

The display unit 40 is a device that is constituted by, for example, a liquid crystal display, an organic EL display, or the like and that displays the analysis result.

The analysis result of the fatigue and/or stress state of the organization, described with reference to FIG. 4, may be displayed in the form of a pie chart as illustrated in FIG. 9, for example, and may be displayed on the display unit 40. In this case, the fatigue and/or stress state in the entirety of the organization is easier to grasp. Moreover, a bias in distribution of the fatigue and/or stress values of the members belonging to the organization can be visually grasped.

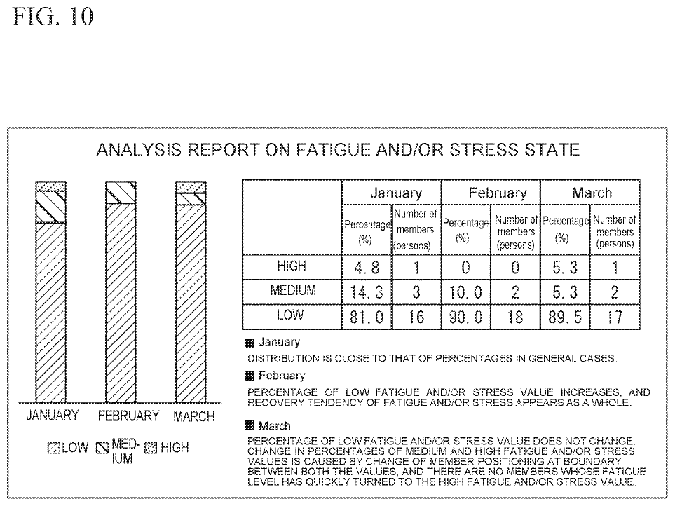

FIG. 10 is a chart representing another example of the analysis result displayed on the display unit 40 of the analysis system 1A of FIG. 8. As illustrated in FIG. 10, the fatigue and/or stress state of the organization may be displayed in the form of a bar graph per month. Comparison per month is made easier by displaying the fatigue and/or stress state in the bar graph. An influence of the fatigue and/or stress state upon performance of the organization can be distinguished by comparing the fatigue and/or stress state of the organization per month with the performance of the organization per month. The display in the bar graph further makes easier comparison of the fatigue and/or stress state between different organizations.

### Embodiment 2

An analysis system according to Embodiment 2 of the present disclosure is described. Embodiment 2 is described mainly on different points from Embodiment 1. In the following description of Embodiment 2, the same or similar components as or to those in Embodiment 1 are denoted by the same reference signs. Moreover, description overlapping with that of Embodiment 1 is omitted in Embodiment 2.

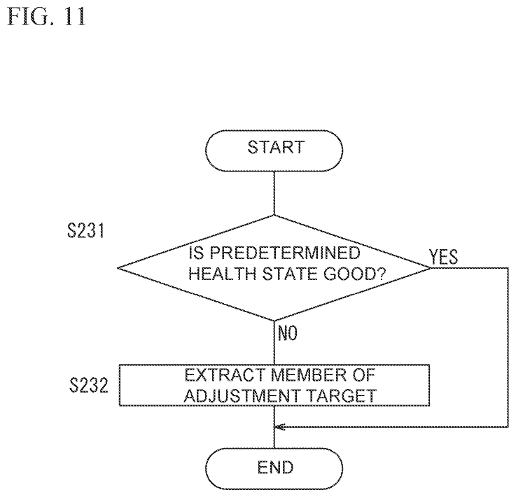

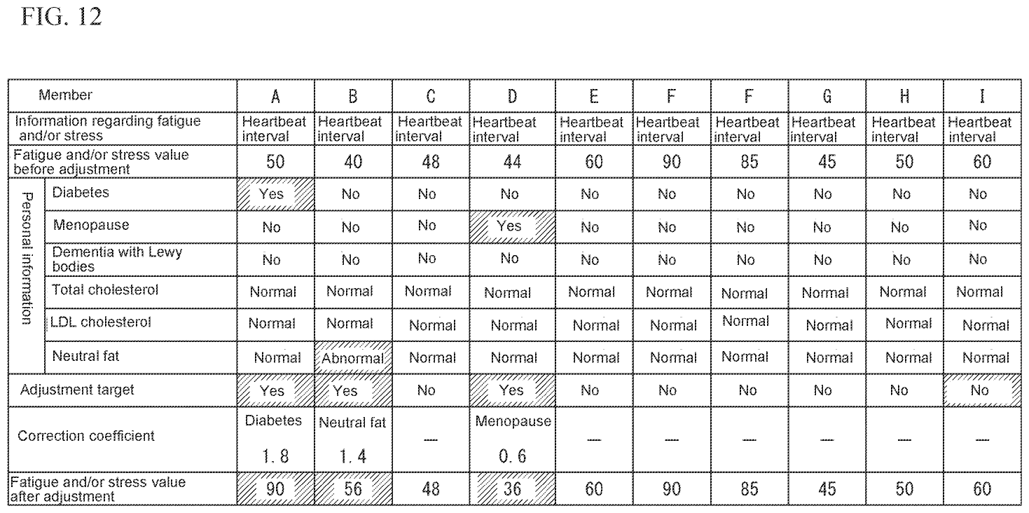

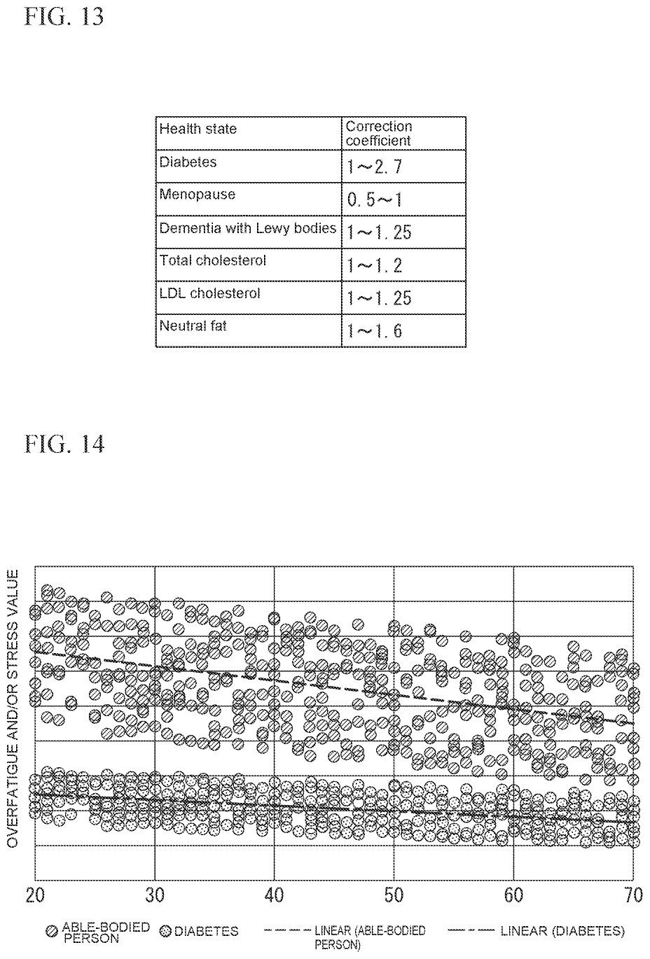

FIG. 11 is a flowchart illustrating steps of extracting a member of the adjustment target in the analysis method according to Embodiment 2. FIG. 12 is a table indicating the fatigue and/or stress values of the individual members A to I belonging to the organization and personal information of the members. FIG. 13 is a table indicating a correction coefficient for each of different health states.

Embodiment 2 is different from Embodiment 1 in correcting the fatigue and/or stress value of the member of the adjustment target.

For instance, when any member has a chronic disease, such as diabetes, possibly affecting the autonomic nerve index, the fatigue and/or stress value of the member may be affected for a long period. In consideration of the above point, the fatigue and/or stress state of the organization can be analyzed with higher accuracy by, instead of excluding the fatigue and/or stress value of the member in question from the analysis target, correcting the fatigue and/or stress value depending on the kind of the disease and the severity of the disease.

In this embodiment, when the predetermined health state included in the health information is not good, the adjustment target extraction unit 32 in the control unit 30 extracts the member in question as the adjustment target. The extraction of the member of the adjustment target in this embodiment is described with reference to FIG. 11.

The adjustment target extraction unit 32 in the control unit 30 determines whether the predetermined health state is good (step S231). Whether the predetermined health state is good can be determined depending on whether a member is in the predetermined heath state, namely whether a member has a predetermined disease such as diabetes, menopause, or dementia with Lewy bodies, as illustrated in an example of FIG. 13. Alternatively, whether the predetermined health state is good can also be determined depending on whether numerical values of test results on, for example, total cholesterol, LDL cholesterol, and neutral fat, are normal or abnormal. The numerical values of the test results include, for example, weight, judgement of metabolic syndrome by waist circumference, eyesight, hearing ability (1 kHz, 4 kHz, or conversation method), blood pressure (systolic/diastolic), urinalysis (urine protein/urinary occult blood), liver function (AST, ALT, and γ-GTP), lipid (LDL cholesterol, HDL cholesterol, total cholesterol, triglyceride, and non-HDL cholesterol), metabolism (uric acid), glucose metabolism (fasting blood glucose level, blood glucose level immediately after meal, blood glucose level after meal, random blood glucose level, and HbA1c), blood cell (white blood cell count, red blood cell count, hemoglobin, hematocrit, and platelet count), fecal occult blood, PSA, or results of internal medical examination. Whether the test results are normal or abnormal is determined depending on, for example, whether the numerical values of the test results are within or outside normal reference ranges and is previously stored as the personal information in the information storage unit 20. In the example of FIG. 13, because the member A has diabetes as a disease, the determination that the predetermined health state is not good is made on the member A. Similarly, in the example of FIG. 13, because the test result of neutral fat on the member B is abnormal and the member D has menopause as a disease, the determination that the predetermined health state is not good is also made on both the members B and D.

If the predetermined health state is determined to be not good (No in step S231), the member in question is extracted as the member of the adjustment target by the adjustment target extraction unit 32 (step S232). If the predetermined health state is determined to be good (Yes in step S231), the steps of extracting the member of the adjustment target is ended.

If the predetermined health state is determined to be not good (No in step S231), the adjustment target extraction unit 32 extracts the member in question as the member of the adjustment target (step S232).

After the extraction of the member of the adjustment target, the set of the fatigue and/or stress values is adjusted by the fatigue and/or stress value adjustment unit 33 (step S15 in FIG. 5).

The fatigue and/or stress value adjustment unit 33 corrects the fatigue and/or stress value of the member of the adjustment target. The fatigue and/or stress value is corrected in accordance with the correction coefficient denoted, by way of example, in FIG. 13. For instance, when the fatigue and/or stress value denoted in FIG. 12 indicates the total power, the fatigue and/or stress value of the member A with diabetes is corrected by using the correction coefficient for diabetes in FIG. 13. The correction coefficient for diabetes is denoted in a range of 1 to 2.7. This indicates that different correction coefficients can be used depending on the severity of diabetes. In the example of FIG. 12, the correction coefficient of 1.8 is applied to the member A depending on the severity of diabetes. When a member has diabetes, there is a tendency that a total power value of the member is calculated as a lower value than that of an able-bodied person. In consideration of such a tendency, the fatigue and/or stress value of the member A is multiplied by the correction coefficient of 1.8 to correct the fatigue and/or stress value. Note that, when the fatigue and/or stress level is represented in terms of the total power, a smaller value indicates a higher fatigue and/or stress level. For that reason, when the fatigue and/or stress value is calculated as a lower value attributable to diabetes like the member A, the fatigue and/or stress value is corrected to a greater value than the actual fatigue and/or stress value by using the correction coefficient of 1.8, and the corrected value is used in the analysis. By correcting the fatigue and/or stress value depending on the health state, a deviation of the fatigue and/or stress value caused by the health state of the member can be corrected, and the analysis on the fatigue and/or stress state of the organization can be performed with higher accuracy. The fatigue and/or stress value of the member B is corrected by using the correction coefficient of 1.4, and the fatigue and/or stress value of the member D is corrected by using the correction coefficient of 0.6. Note that the correction coefficients listed in FIG. 13 are merely examples and other correction coefficients outside the numeral ranges listed in FIG. 13 can also be used.

### Effects

The following effects can be obtained with Embodiment 2.

The personal information includes the health information of the plurality of members. The predetermined condition includes whether the predetermined health state included in the health information is good. When the predetermined health state is not good, the adjustment target extraction unit 32 in the control unit 30 extracts the member of the adjustment target. The fatigue and/or stress value adjustment unit 33 in the control unit 30 adjusts the set of the fatigue and/or stress values by correcting the fatigue and/or stress value of the member of the adjustment target.

With the configuration described above, when the fatigue and/or stress value is affected for a long period due to the presence of a chronic disease, for example, the fatigue and/or stress value is corrected depending on the type of the disease and so on, whereby the fatigue and/or stress state of the organization can be analyzed with exclusion of the influence not related to the organization. As a result, the analysis accuracy can be improved.

The fatigue and/or stress value adjustment unit 33 in the control unit 30 adjusts the set of the fatigue and/or stress values by correcting the fatigue and/or stress value of the member of the adjustment target in accordance with the correction coefficient for each health state.

With the configuration described above, because of using the correction coefficient for each health state possibly affecting the fatigue and/or stress value, the fatigue and/or stress state of the organization can be analyzed with exclusion of the influence not related to the organization. As a result, the analysis accuracy can be improved.

While, in the above-described embodiment, the presence of diabetes, menopause, or dementia with Lewy bodies and the test results of total cholesterol, LDL cholesterol, and neutral fat are used as the health information, the health information is not limited to those examples. The presence of other various diseases or test results possibly affecting the fatigue and/or stress value can also be used. In addition, the fatigue and/or stress value may be corrected for the member who has experienced a predetermined life event.

While the above embodiment has been described in connection with the example in which the fatigue and/or stress value is corrected in accordance with the correction coefficient for each health state, a method of correcting the fatigue and/or stress value is not limited to the above-mentioned example.

FIG. 14 is a graph indicating an example of statistical data used in the analysis system 1 according to Modification 1 of Embodiment 2. The fatigue and/or stress value adjustment unit 33 in the control unit 30 may correct the fatigue and/or stress value of the member of the adjustment target based on statistical data of the fatigue and/or stress values with respect to the health state instead of using the correction coefficient corresponding to the health state.

In the graph of FIG. 14, data obtained by plotting the magnitudes of the fatigue and/or stress values of able-bodied persons with a horizontal axis indicating age is represented as linear data in accordance with a linear regression analysis, for example. Similarly, data obtained by plotting the magnitudes of the fatigue and/or stress values of diabetes patients with a horizontal axis indicating age is represented as linear data in accordance with a linear regression analysis, for example. The statistical data such as illustrated in FIG. 14 may be prepared for each health state, and the fatigue and/or stress value may be corrected based on the prepared statistical data. The statistical data may be previously acquired by the control unit 30 and may be stored into the storage unit in the control unit 30. For instance, an operator of the analysis system 1 may previously input the statistical data for each health state into the control unit 30. Alternatively, the control unit 30 can also utilize the statistical data stored in an external device through communication with the external device. The statistical data may be acquired by the control unit 30 at the predetermined timing of, for example, once a week, a month, or a year.

For instance, when a member has diabetes, the fatigue and/or stress value may be corrected depending on how far the fatigue and/or stress value of the member is deviated from the linear data for diabetes in the graph of FIG. 14.

In the case of using the statistical data to correct the fatigue and/or stress value, correction accuracy for the fatigue and/or stress value is improved with further accumulation of the data of the fatigue and/or stress values. Accordingly, each time the analysis system 1 is used, the accuracy in analyzing the fatigue and/or stress state can be improved.

### Embodiment 3

An analysis system according to Embodiment 3 of the present disclosure is described. Embodiment 3 is described mainly on different points from Embodiment 1. In the following description of Embodiment 3, the same or similar components as or to those in Embodiment 1 are denoted by the same reference signs. Moreover, description overlapping with that of Embodiment 1 is omitted in Embodiment 3.

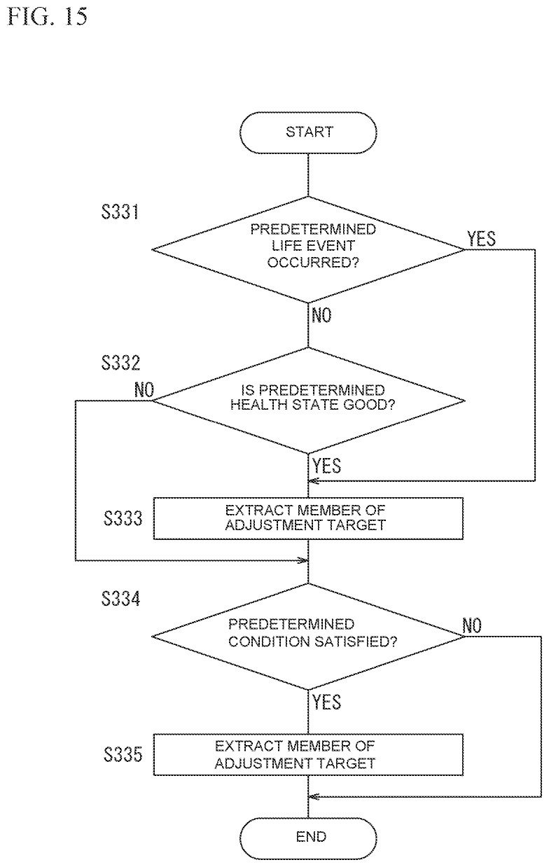

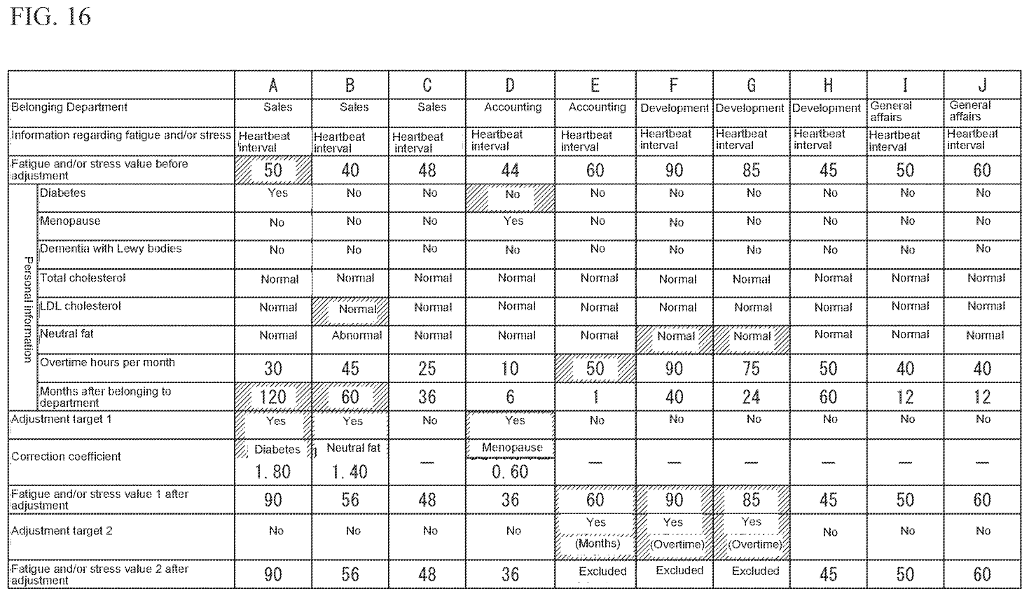

FIG. 15 is a flowchart illustrating steps of extracting a member of the adjustment target in an analysis method according to Embodiment 3. FIG. 16 is a table indicating the fatigue and/or stress values of the individual members A to I belonging to the organization and personal information of the members.

Embodiment 3 is different from Embodiment 1 in that the personal information includes information regarding work in the organization. Furthermore, Embodiment 3 is different from Embodiment 1 in that the adjustment target extraction unit 32 in the control unit 30 extracts the member of the adjustment target based on the information regarding the work in the organization.

The fatigue and/or stress value of the member may be affected by factors related to the work in the organization, such as a lot of overtime hours, immediately after personnel change, and many long-distance business trips, for example. In trying to analyze a fatigue and/or stress state affected by a particular one among the factors related to the work in the organization, it is desirable to exclude the influence of other factors than the particular one upon the fatigue and/or stress value. In this embodiment, to exclude the influence of the other factors than the particular one, the fatigue and/or stress values of the members who have the other factors than the particular one are corrected or excluded from the analysis.

Steps of extracting the member of the adjustment target in Embodiment 3 is described with reference to FIG. 15. Note that steps S331 to S333 in FIG. 15 are similar to the steps S131 to S133 in FIG. 1 described in Embodiment 1 and hence description of those steps is omitted here.

In the steps S331 to S333, the adjustment target extraction unit 32 in the control unit 30 extracts the member of the adjustment target based on the health information of the member or the life event occurred on the member. In the example of FIG. 16, the members A, B and D for whom “Yes” is denoted in the column of “Adjustment target 1” are extracted as the adjustment targets.

Then, the adjustment target extraction unit 32 in the control unit 30 determines whether an item in the personal information, the item being related to the work in the organization, satisfies the predetermined condition (step S334). The predetermined condition includes, for example, whether the overtime hours exceed a predetermined threshold, whether a predetermined period has elapsed from the personnel change, or whether the number of long-distance business trips exceed a predetermined threshold.

If the information related to the organization satisfies the predetermined condition (Yes in step S334), the adjustment target extraction unit 32 in the control unit 30 extracts the member in question as the member of the adjustment target (step S335). If the information related to the organization does not satisfy the predetermined condition (No in step S334), the steps of extracting the member of the adjustment target is ended.

In this embodiment, it is assumed that the predetermined condition is satisfied when the overtime hours per month exceed 67.5 hours and when the number of months after belonging to the current department is not more than 3 months. In the above case, as illustrated in FIG. 16, the members E, F and G for whom “Yes” is denoted in the column of “Adjustment target 2” are extracted as the adjustment targets.

After the extraction of the members of the adjustment targets, the fatigue and/or stress value adjustment unit 33 in the control unit 30 adjusts the set of the fatigue and/or stress values by correcting the fatigue and/or stress values of the members of the adjustment targets or by excluding the fatigue and/or stress values thereof from the analysis (step S15 in FIG. 5).

For the members extracted in step S333 of FIG. 15, the fatigue and/or stress values are corrected by using the correction coefficients. As a result, the fatigue and/or stress values of the members A, B and D are corrected, and the set of the fatigue and/or stress values is adjusted to take values denoted in the column “Fatigue and/or stress value 1 after adjustment” in FIG. 15.

For the members extracted in step S335 of FIG. 15, the fatigue and/or stress values are excluded from the analysis target. As a result, the fatigue and/or stress values of the members E, F and G are excluded from among the values denoted in the column “Fatigue and/or stress value 1 after adjustment”, and the set of the fatigue and/or stress values is adjusted to include values denoted in the column “Fatigue and/or stress value 2 after adjustment”.

### Effects

The following effects can be obtained with Embodiment 3.

The personal information includes the information regarding the work in the organization. The adjustment target extraction unit 32 in the control unit 30 extracts the member of the adjustment target based on the information regarding the work in the organization. The fatigue and/or stress value adjustment unit 33 in the control unit 30 adjusts the set of the fatigue and/or stress values by correcting the fatigue and/or stress value of the member of the adjustment target or by excluding the fatigue and/or stress value thereof from the analysis.

With the configuration described above, the fatigue and/or stress state of the organization can be analyzed with exclusion of predetermined one or more among the factors that are related to the work in the organization and that deteriorate the fatigue and/or stress value. Accordingly, appropriate action against the deterioration of the fatigue and/or stress value can be promoted. Thus, the fatigue and/or stress state of the organization can be analyzed with the exclusion of the influence of the predetermined factors, for example, the overtime hours and the personnel change.

While the above embodiment has been described in connection with the example in which the fatigue and/or stress value is excluded from the analysis target when the information regarding the work in the organization satisfies the predetermined condition, the present disclosure is not limited to the above-mentioned case. When the information regarding the work in the organization satisfies the predetermined condition, the fatigue and/or stress value may be corrected. In an example, the fatigue and/or stress value may be corrected depending on the overtime hours.

### Embodiment 4

An analysis system according to Embodiment 4 of the present disclosure is described. Embodiment 4 is described mainly on different points from Embodiment 1. In the following description of Embodiment 4, the same or similar components as or to those in Embodiment 1 are denoted by the same reference signs. Moreover, description overlapping with that of Embodiment 1 is omitted in Embodiment 4.

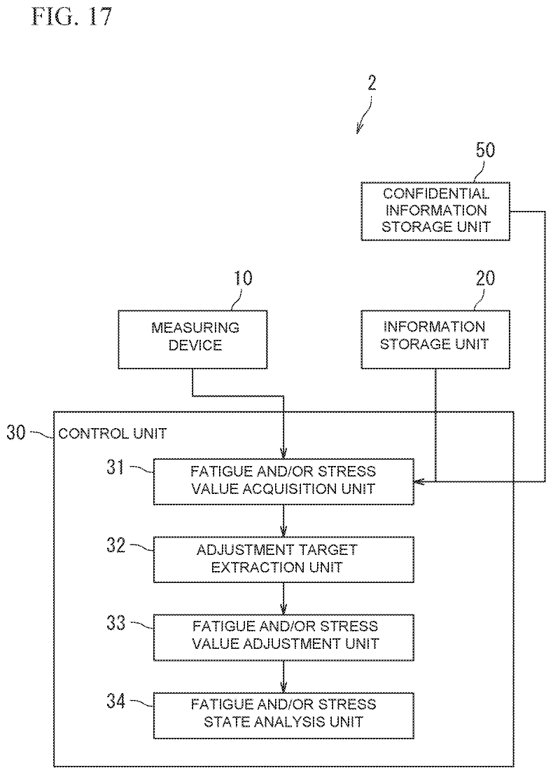

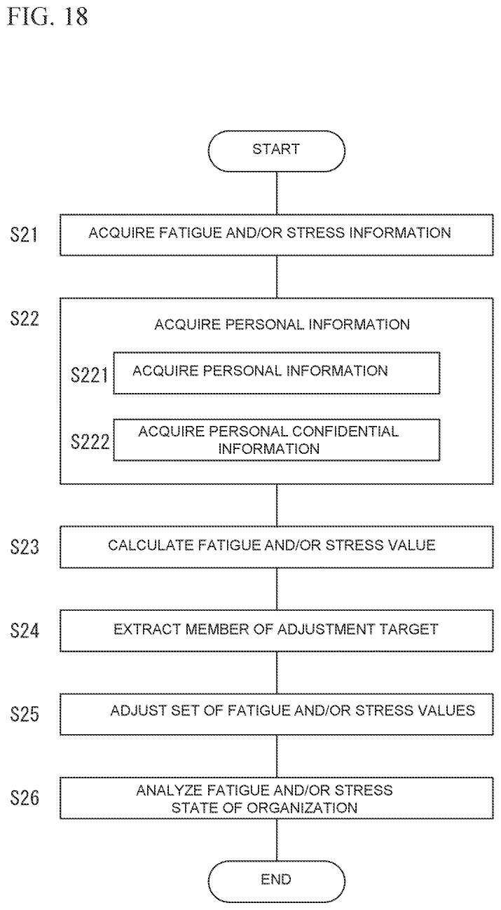

FIG. 17 is a block diagram illustrating an analysis system 2 according to Embodiment 4. FIG. 18 is a flowchart illustrating an analysis method in the analysis system 2 of FIG. 17.

As illustrated in FIG. 17, Embodiment 4 is different from Embodiment 1 in that the analysis system 2 includes a confidential information storage unit 50.

The confidential information storage unit 50 stores personal confidential information that is kept confidential to the organization. The personal confidential information includes private information of the member to be not known to the organization, such as the health information of the member in the past (before belonging to the organization) or information regarding the life events not handled by the organization. The information regarding the life events not handled by the organization is the private information of the member regarding, for example, the loss of a friend or an acquaintance, or the loss of a pet, and is information that is not obligated to report to the organization. The loss of a friend or an acquaintance includes the loss of a partner in terms of partnership (such as common-law marriage or partnership in sexual minority) except for law marriage. The personal confidential information is entered by the member oneself and is stored into confidential information storage unit 50.

The confidential information can be entered with, for example, an electronic device, such as a PC, a tablet, or a smartphone owned by the member oneself.

The confidential information storage unit 50 is constituted by a memory, a storage device such as HDD (Hard Disk Drive) or SSD (Solid State Drive), or the like.

The confidential information storage unit 50 may be constituted by hardware different from the information storage unit 20. Alternatively, the confidential information storage unit 50 may be constituted on the same hardware as that of the information storage unit 20. In the latter case, the access right to the confidential information storage unit 50 is to be controlled such that the confidential information cannot be referred to by any other person than the member oneself having entered the confidential information.

An analysis method in the analysis system 2 is described with reference to FIG. 18. This embodiment is different from Embodiment 1 in a step of acquiring the personal information, namely a step S22, and a step of extracting the member of the adjustment target, namely a step S24. Steps S21, S23, and S25 to S26 are the same as S11, S13, and S15 to S16 in Embodiment 1, respectively, and hence description of those steps is omitted here.

In the step S22, the control unit 30 acquires multiple types of personal information. First, the control unit 30 acquires the personal information other than the confidential information (step S221). The control unit 30 acquires the personal information by referring to the personal information stored in the information storage unit 20 and by storing the personal information into the storage unit in the control unit 30.

Then, the control unit 30 acquires the personal confidential information (step S222). The control unit 30 acquires the personal confidential information by referring to the personal confidential information stored in the confidential information storage unit 50 and by storing the personal confidential information into the storage unit in the control unit 30.

The control unit 30 analyzes the fatigue and/or stress state of the organization based on the personal information stored in the information storage unit 20 and the personal confidential information stored in the confidential information storage unit 50. In more detail, in the step S24, the adjustment target extraction unit 32 in the control unit 30 extracts the member of the adjustment target based on the personal information and the personal confidential information. As in the case of referring to the personal information, the adjustment target extraction unit 32 can extract the employee who becomes the target for correction or exclusion, by referring to the personal confidential information.

### Effects

The following effects can be obtained with Embodiment 4.

The analysis system 2 includes the confidential information storage unit 50. The confidential information storage unit 50 stores the personal confidential information that is kept confidential to the organization. The control unit 30 analyzes the fatigue and/or stress state of the organization based on the personal information stored in the information storage unit 20 and the personal confidential information stored in the confidential information storage unit 50.

With the configuration described above, the fatigue and/or stress state of the organization can be analyzed taking into consideration the personal information of the member as well which is to be kept confidential to the organization. Accordingly, the analysis accuracy can be improved.

The confidential information storage unit 50 may be included in the information storage unit 20. In an example, the confidential information storage unit 50 can be realized by controlling the access right to the confidential information storage unit 50 such that the confidential information stored in the confidential information storage unit 50 is not disclosed to the organization.

### Embodiment 5

An analysis system according to Embodiment 5 of the present disclosure is described. Embodiment 5 is described mainly on different points from Embodiment 1. In the following description of Embodiment 5, the same or similar components as or to those in Embodiment 1 are denoted by the same reference signs. Moreover, description overlapping with that of Embodiment 1 is omitted in Embodiment 5.

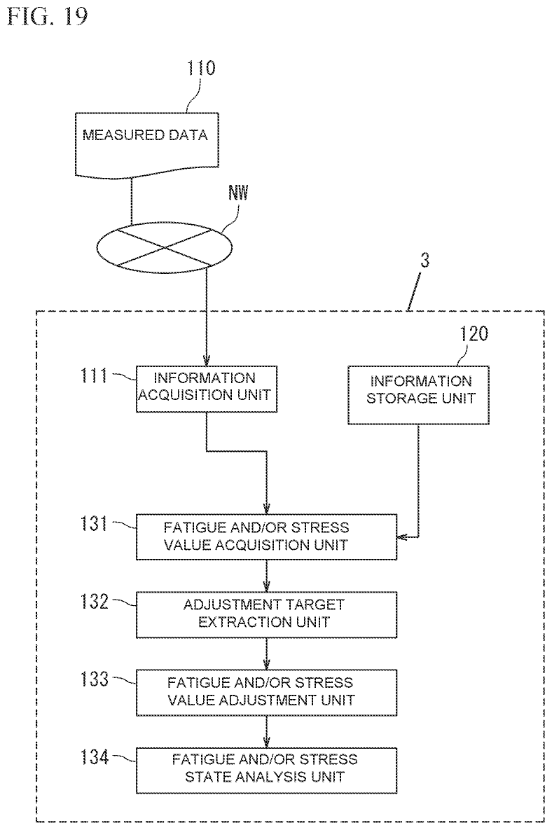

FIG. 19 is a block diagram illustrating a configuration of an analysis system 3 according to Embodiment 5. As illustrated in FIG. 19, Embodiment 5 is different from Embodiment 1 in that the analysis system 3 includes an information acquisition unit 111. Moreover, Embodiment 5 is different from Embodiment 1 in that a fatigue and/or stress value acquisition unit 131, an adjustment target extraction unit 132, a fatigue and/or stress value adjustment unit 133, a fatigue and/or stress state analysis unit 134, and an information storage unit 120 are constituted by a plurality of devices.

The information acquisition unit 111 is constituted by, for example, a communication circuit and, as illustrated in FIG. 19, acquires measured data 110 regarding the fatigue and/or the stress of the member via a network NW. The measured data 110 can be acquired by, for example, a biological sensor that is a device different from the analysis system 3. The analysis system 3 can acquire the measured data 110 by the information acquisition unit 111 via the network, for example, and can store the measured data 110 into, for example, a database in the analysis system 3. In this embodiment, since the analysis system 3 includes the information acquisition unit 111, the fatigue and/or stress state of the organization can be analyzed by using the measured data 110 that has been acquired by, for example, the biological sensor outside the analysis system 3.

Furthermore, in this embodiment, the fatigue and/or stress value acquisition unit 131, the adjustment target extraction unit 132, the fatigue and/or stress value adjustment unit 133, and the fatigue and/or stress state analysis unit 134 are constituted by devices different from one another. Here, each of the devices has a configuration including any of various processors, such as CPU, MPU, CPU, FPGA, DSP, and ASIC, for example. Functions of the fatigue and/or stress value acquisition unit 131, the adjustment target extraction unit 132, the fatigue and/or stress value adjustment unit 133, and the fatigue and/or stress state analysis unit 134 are executed by the processors in the individual devices. In this case, the fatigue and/or stress value acquisition unit 131, the adjustment target extraction unit 132, the fatigue and/or stress value adjustment unit 133, and the fatigue and/or stress state analysis unit 134 can communicate with one another via the network. Since the fatigue and/or stress value acquisition unit 131, the adjustment target extraction unit 132, the fatigue and/or stress value adjustment unit 133, and the fatigue and/or stress state analysis unit 134 are constituted by the devices different from one another, components of the individual devices can be disposed in physically different places.

The fatigue and/or stress value acquisition unit 131 calculates the fatigue and/or stress value for each of the members based on the information regarding the fatigue and/or the stress of the member, that information having been received by the information acquisition unit 111. The processor constituting the fatigue and/or stress value acquisition unit 131 may include a storage device (not illustrated), such as HDD or SSD, for example. The information regarding the fatigue and/or the stress, acquired by the information acquisition unit 111, is stored into the storage device, and the fatigue and/or stress value acquisition unit 131 can calculate the fatigue and/or stress value by using the information regarding the fatigue and/or the stress which has been stored in the storage device.

### Effects

The following effects can be obtained with Embodiment 5.

The analysis system 3 is an analysis system for analyzing a fatigue and/or stress state of an organization to which a plurality of members belongs. The analysis system 3 includes the information acquisition unit 111, the information storage unit 120, the fatigue and/or stress value acquisition unit 131, the adjustment target extraction unit 132, the fatigue and/or stress value adjustment unit 133, and the fatigue and/or stress state analysis unit 134. The information acquisition unit 111 acquires the information regarding the fatigue and/or the stress of the plurality of members. The information storage unit 120 stores the personal information of the plurality of members. The fatigue and/or stress value acquisition unit 131 calculates the fatigue and/or stress value for each of the plurality of members based on the information regarding the fatigue and/or the stress. The adjustment target extraction unit 132 extracts the member of the adjustment target from among the plurality of members based on the personal information and the predetermined condition for extracting the member of the adjustment target. The fatigue and/or stress value adjustment unit 133 adjusts the set of the fatigue and/or stress values by correcting the fatigue and/or stress value of the member of the adjustment target or by excluding the fatigue and/or stress value thereof from the analysis. The fatigue and/or stress state analysis unit 134 analyzes the fatigue and/or stress state of the organization based on the adjusted set of the fatigue and/or stress values.

With the configuration described above, the analysis system 3 can be provided which has succeeded in improving the accuracy in analyzing the fatigue and/or stress state of the organization. Furthermore, the respective functions of the fatigue and/or stress value acquisition unit 131, the adjustment target extraction unit 132, the fatigue and/or stress value adjustment unit 133, and the fatigue and/or stress state analysis unit 134 can be realized with the different processors. Accordingly, the components of the individual units can be disposed in physically different places.

While the above embodiment has been described in connection with the example in which the fatigue and/or stress value acquisition unit 131, the adjustment target extraction unit 132, the fatigue and/or stress value adjustment unit 133, and the fatigue and/or stress state analysis unit 134 are constituted by the devices different from one another, the present disclosure is not limited to the above-mentioned example. In another example, two or more among the fatigue and/or stress value acquisition unit 131, the adjustment target extraction unit 132, the fatigue and/or stress value adjustment unit 133, and the fatigue and/or stress state analysis unit 134 may be constituted by one device. Furthermore, the information storage unit 120 may be constituted by a device different from the fatigue and/or stress value acquisition unit 131, the adjustment target extraction unit 132, the fatigue and/or stress value adjustment unit 133, and the fatigue and/or stress state analysis unit 134. In other words, the fatigue and/or stress value acquisition unit 131, the adjustment target extraction unit 132, the fatigue and/or stress value adjustment unit 133, the fatigue and/or stress state analysis unit 134, and the information storage unit 120 may be constituted by devices different from one another or by the same device for all or some of those units.

### OVERVIEW OF EMBODIMENTS

(1) The present disclosure provides an analysis system for analyzing a fatigue and/or stress state of an organization to which a plurality of members belongs, the analysis system comprising an information acquisition unit configured to acquire information regarding fatigue and/or stress of the plurality of members; an information storage unit configured to store personal information of the plurality of members; a fatigue and/or stress value acquisition unit configured to acquire a fatigue and/or stress value for each of the plurality of members, the fatigue and/or stress value being calculated based on the information regarding the fatigue and/or the stress; an adjustment target extraction unit configured to extract the member of an adjustment target from among the plurality of members based on the personal information including health information of the plurality of members and a predetermined condition for extracting the member of the adjustment target, the predetermined condition including whether a predetermined health state included in the health information is good, a fatigue and/or stress value adjustment unit configured to adjust a set of the fatigue and/or stress values by correcting the fatigue and/or stress value of the member of the adjustment target or by excluding the fatigue and/or stress value thereof from an analysis; and a fatigue and/or stress state analysis unit configured to analyze the fatigue and/or stress state of the organization based on an adjusted set of the fatigue and/or stress values.

With the configuration described above, the analysis system can be provided which has succeeded in improving the accuracy in analyzing the fatigue and/or stress state of the organization.

(2) In the analysis system stated in (1), the adjustment target extraction unit may extract the member of the adjustment target when the predetermined health state is not good, and the fatigue and/or stress value adjustment unit may adjust the set of the fatigue and/or stress values by excluding the fatigue and/or stress value of the member of the adjustment target from the analysis.

With the configuration described above, the influence of factors deteriorating the fatigue and/or stress value, the factors being not related to the operations of the organization, can be excluded, and the accuracy in analyzing the fatigue and/or stress state of the organization can be improved.

(3) The present disclosure provides an analysis system for analyzing a fatigue and/or stress state of an organization to which a plurality of members belongs, the analysis system comprising an information acquisition unit configured to acquire information regarding fatigue and/or stress of the plurality of members; an information storage unit configured to store personal information of the plurality of members; a fatigue and/or stress value acquisition unit configured to acquire a fatigue and/or stress value for each of the plurality of members, the fatigue and/or stress value being calculated based on the information regarding the fatigue and/or the stress; an adjustment target extraction unit configured to extract the member of an adjustment target from among the plurality of members based on the personal information including life events occurred on the plurality of members and a predetermined condition for extracting the member of the adjustment target, the predetermined condition including whether a predetermined life event has occurred; a fatigue and/or stress value adjustment unit configured to adjust a set of the fatigue and/or stress values by correcting the fatigue and/or stress value of the member of the adjustment target or by excluding the fatigue and/or stress value thereof from an analysis; and a fatigue and/or stress state analysis unit configured to analyze the fatigue and/or stress state of the organization based on an adjusted set of the fatigue and/or stress values.

With the configuration described above, the analysis system can be provided which has succeeded in improving the accuracy in analyzing the fatigue and/or stress state of the organization.

(4) In the analysis system stated in (3), the adjustment target extraction unit may extract the member of the adjustment target when the predetermined life event has occurred, and the fatigue and/or stress value adjustment unit may adjust the set of the fatigue and/or stress values by excluding the fatigue and/or stress value of the member of the adjustment target from the analysis.

With the configuration described above, the influence of factors deteriorating the fatigue and/or stress value, the factors being not related to the operations of the organization, can be excluded, and the accuracy in analyzing the fatigue and/or stress state of the organization can be improved.

(5) In the analysis system stated in (4), the predetermined life event may include an event occurred during a period from a past predetermined time to an acquisition time of the information regarding the fatigue and/or the stress.

With the configuration described above, since such a life event, for example, that is estimated not to take part in deteriorating the fatigue and/or stress value at the present time is regarded as a factor causing noise and is not taken into consideration, the analysis accuracy can be further improved.

(6) In the analysis system stated in any one of (1) to (5), the personal information may include the health information of the plurality of members, the predetermined condition may include whether the predetermined health state included in the health information is good, the adjustment target extraction unit may extract the member of the adjustment target when the predetermined health state is not good, and the fatigue and/or stress value adjustment unit may adjust the set of the fatigue and/or stress values by correcting the fatigue and/or stress value of the member of the adjustment target.

With the configuration described above, when the fatigue and/or stress value is affected for a long period due to the presence of a chronic disease, for example, the fatigue and/or stress value is corrected depending on the type of the disease and so on, whereby the fatigue and/or stress state of the organization can be analyzed with exclusion of the influence not related to the organization. Therefore, the analysis accuracy can be improved.

(7) In the analysis system stated in (6), the fatigue and/or stress value adjustment unit may adjust the set of the fatigue and/or stress values by correcting the fatigue and/or stress value of the member of the adjustment target in accordance with a correction coefficient for the predetermined health state.

With the configuration described above, since the correction coefficient is used for the health state possibly affecting the fatigue and/or stress value, the fatigue and/or stress state of the organization can be analyzed with exclusion of the influence not related to the organization. Therefore, the analysis accuracy can be improved.

(8) In the analysis system stated in (6), the fatigue and/or stress value adjustment unit may adjust the set of the fatigue and/or stress values by correcting the fatigue and/or stress value of the member of the adjustment target based on statistical data of the fatigue and/or stress values for the predetermined health state, the statistical data being acquired in advance.

With the configuration described above, since the statistical data of the fatigue and/or stress values for the health state can be accumulated, the correction accuracy can be improved with an increase in amount of the measured data.

(9) In the analysis system stated in any one of (1) to (8), the personal information may include information regarding work in the organization, the adjustment target extraction unit may extract the member of the adjustment target based on the information regarding the work in the organization and may adjust the set of the fatigue and/or stress values by correcting the fatigue and/or stress value of the member of the adjustment target or by excluding the fatigue and/or stress value thereof from the analysis.

With the configuration described above, the fatigue and/or stress state of the organization can be analyzed with exclusion of predetermined one or more among the factors that are related to the operations of the organization and that deteriorate the fatigue and/or stress value. Accordingly, appropriate action against the deterioration of the fatigue and/or stress value can be promoted.

(10) The analysis system stated in any one of (1) to (9) may further comprise a confidential information storage unit configured to store personal confidential information that is kept confidential to the organization, and the fatigue and/or stress state analysis unit may analyze the fatigue and/or stress state of the organization based on the personal information stored in the information storage unit and the personal confidential information stored in the confidential information storage unit.

With the configuration described above, the fatigue and/or stress state of the organization can be analyzed while the personal information of the member which is to be kept confidential to the organization is also taken into consideration. Therefore, the analysis accuracy can be improved.

(11) In the analysis system stated in any one of (1) to (10), the fatigue and/or stress state analysis unit may analyze the fatigue and/or stress state of the organization by classifying the fatigue and/or stress values into a plurality of categories depending on magnitudes of the fatigue and/or stress values.

With the configuration described above, the fatigue and/or stress state in the entirety of the organization can be easily grasped.

(12) The analysis system stated in (11) may further comprise a display unit configured to display an analysis result of the fatigue and/or stress state of the organization, and the analysis result may be graphically displayed on the display unit.

With the configuration described above, the fatigue and/or stress state of the organization can be displayed in a visually easily recognizable manner.

(13) In the analysis system stated in any one of (1) to (12), the information regarding the fatigue and/or the stress may include biological information of the plurality of members.

With the configuration described above, the fatigue and/or stress state can be analyzed by using information that is obtained from the biological information, such as the autonomic nerve index, for example.

(14) The present disclosure provides an analysis device for analyzing a fatigue and/or stress state of an organization to which a plurality of members belongs, the analysis device comprising a fatigue and/or stress value acquisition unit configured to acquire a fatigue and/or stress value for each of the plurality of members, the fatigue and/or stress value being calculated based on information regarding fatigue and/or stress of the plurality of members; an adjustment target extraction unit configured to acquire the member of an adjustment target from among the plurality of members, the member of the adjustment target being extracted based on personal information of the plurality of members, the personal information including health information of the plurality of members, and on a predetermined condition for extracting the member of the adjustment target, the predetermined condition including whether a predetermined health state included in the health information is good; a fatigue and/or stress value adjustment unit configured to acquire a set of the fatigue and/or stress values, the set of the fatigue and/or stress values being adjusted by correcting the fatigue and/or stress value of the member of the adjustment target or by excluding the fatigue and/or stress value thereof from an analysis; and a fatigue and/or stress state analysis unit configured to analyze the fatigue and/or stress state of the organization based on the adjusted set of the fatigue and/or stress values.

With the configuration described above, the analysis device can be provided which has succeeded in improving the accuracy in analyzing the fatigue and/or stress state of the organization.

(15) The present disclosure provides an analysis device for analyzing a fatigue and/or stress state of an organization to which a plurality of members belongs, the analysis device comprising a fatigue and/or stress value acquisition unit configured to acquire a fatigue and/or stress value for each of the plurality of members, the fatigue and/or stress value being calculated based on information regarding fatigue and/or stress of the plurality of members; an adjustment target extraction unit configured to acquire the member of an adjustment target from among the plurality of members, the member of the adjustment target being extracted based on personal information of the plurality of members, the personal information including life events occurred on the plurality of members, and on a predetermined condition for extracting the member of the adjustment target, the predetermined condition including whether a predetermined life event has occurred; a fatigue and/or stress value adjustment unit configured to acquire a set of the fatigue and/or stress values, the set of the fatigue and/or stress values being adjusted by correcting the fatigue and/or stress value of the member of the adjustment target or by excluding the fatigue and/or stress value thereof from an analysis; and a fatigue and/or stress state analysis unit configured to analyze the fatigue and/or stress state of the organization based on the adjusted set of the fatigue and/or stress values.

With the configuration described above, the analysis device can be provided which has succeeded in improving the accuracy in analyzing the fatigue and/or stress state of the organization.

(16) In the analysis device stated in (14) or (15), at least one of the fatigue and/or stress value acquisition unit, the adjustment target extraction unit, or the fatigue and/or stress value adjustment unit may be constituted by a communication circuit and may receive information from an external device.

With the configuration described above, the fatigue and/or stress value acquisition unit, the adjustment target extraction unit, the fatigue and/or stress value adjustment unit, and the fatigue and/or stress state analysis unit can be individually constituted by different servers or the likes.

(17) The present disclosure provides an analysis method for analyzing a fatigue and/or stress state of an organization to which a plurality of members belongs, the analysis method comprising the steps of acquiring information regarding fatigue and/or stress of the plurality of members; acquiring personal information of the plurality of members; calculating a fatigue and/or stress value for each of the plurality of members based on the information regarding the fatigue and/or the stress; extracting the member of an adjustment target from among the plurality of members based on the personal information including health information of the plurality of members and a predetermined condition for extracting the member of the adjustment target, the predetermined condition including whether a predetermined health state included in the health information is good; adjusting a set of the fatigue and/or stress values by correcting the fatigue and/or stress value of the member of the adjustment target or by excluding the fatigue and/or stress value thereof from an analysis on condition that the personal information takes a predetermined value; and analyzing the fatigue and/or stress state of the organization based on an adjusted set of the fatigue and/or stress values.

With the configuration described above, the analysis method can be provided which has succeeded in improving the accuracy in analyzing the fatigue and/or stress state of the organization.

(18) The present disclosure provides an analysis method for analyzing a fatigue and/or stress state of an organization to which a plurality of members belongs, the analysis method comprising the steps of acquiring information regarding fatigue and/or stress of the plurality of members; acquiring personal information of the plurality of members; calculating a fatigue and/or stress value for each of the plurality of members based on the information regarding the fatigue and/or the stress; extracting the member of an adjustment target from among the plurality of members based on the personal information including life events occurred on the plurality of members and a predetermined condition for extracting the member of the adjustment target, the predetermined condition including whether a predetermined life event has occurred; adjusting a set of the fatigue and/or stress values by correcting the fatigue and/or stress value of the member of the adjustment target or by excluding the fatigue and/or stress value thereof from an analysis on condition that the personal information takes a predetermined value; and analyzing the fatigue and/or stress state of the organization based on an adjusted set of the fatigue and/or stress values.

With the configuration described above, the analysis method can be provided which has succeeded in improving the accuracy in analyzing the fatigue and/or stress state of the organization.

(19) The present disclosure provides a program causing a computer to execute the analysis method stated in (17) or (18).

(20) The present disclosure provides a recording medium on which the program stated in (19) is recorded.

The analysis system, the analysis device, and the analysis method according to the present disclosure are usefully applied to analyze a fatigue and/or stress state of an organization to which a plurality of members belongs.

What is claimed is:

1. An analysis system for analyzing a fatigue and/or stress state of an organization to which a plurality of members belongs, the analysis system comprising:
at least one circuit configured to acquire information regarding fatigue and/or stress of the plurality of members;
at least one memory or storage device configured to store personal information of the plurality of members, the personal information comprising health information; and
at least one processor configured to:
acquire a fatigue and/or stress value for each of the plurality of members, the fatigue and/or stress value being calculated based on the information regarding the fatigue and/or the stress;
extract the member of an adjustment target from among the plurality of members based on the personal information and a predetermined condition for extracting the member of the adjustment target, the predetermined condition comprising whether a predetermined health state in the health information is good;
adjust a set of the fatigue and/or stress values by correcting the fatigue and/or stress value of the member of the adjustment target or by excluding the fatigue and/or stress value thereof from an analysis; and
analyze the fatigue and/or stress state of the organization based on an adjusted set of the fatigue and/or stress values.

2. The analysis system according to claim 1,
wherein the processor is configured to extract the member of the adjustment target when the predetermined health state is not good, and
wherein the processor is configured to adjust the set of the fatigue and/or stress values by excluding the fatigue and/or stress value of the member of the adjustment target from the analysis.

3. An analysis system for analyzing a fatigue and/or stress state of an organization to which a plurality of members belongs, the analysis system comprising:
at least one circuit configured to acquire information regarding fatigue and/or stress of the plurality of members;
at least one memory or storage device configured to store personal information of the plurality of members, the personal information comprising information about life events; and
at least one processor configured to:
acquire a fatigue and/or stress value for each of the plurality of members, the fatigue and/or stress value being calculated based on the information regarding the fatigue and/or the stress;
extract the member of an adjustment target from among the plurality of members based on the personal information and a predetermined condition for extracting the member of the adjustment target, the predetermined condition comprising whether a predetermined life event has occurred;
adjust a set of the fatigue and/or stress values by correcting the fatigue and/or stress value of the member of the adjustment target or by excluding the fatigue and/or stress value thereof from an analysis; and
analyze the fatigue and/or stress state of the organization based on an adjusted set of the fatigue and/or stress values.

4. The analysis system according to claim 3,
wherein the processor is configured to extract the member of the adjustment target when the predetermined life event has occurred, and
wherein the processor is configured to adjust the set of the fatigue and/or stress values by excluding the fatigue and/or stress value of the member of the adjustment target from the analysis.

5. The analysis system according to claim 4, wherein the predetermined life event comprises an event that occurred between a past predetermined time and an acquisition time of the information regarding the fatigue and/or the stress.

6. The analysis system according to claim 1,
wherein the processor is configured to extract the member of the adjustment target when the predetermined health state is not good, and
wherein the processor is configured to adjust the set of the fatigue and/or stress values by correcting the fatigue and/or stress value of the member of the adjustment target.

7. The analysis system according to claim 6, wherein the processor is configured to adjust the set of the fatigue and/or stress values by correcting the fatigue and/or stress value of the member of the adjustment target in accordance with a correction coefficient for the predetermined health state.

8. The analysis system according to claim 6, wherein the processor is configured to adjust the set of the fatigue and/or stress values by correcting the fatigue and/or stress value of the member of the adjustment target based on statistical data of the fatigue and/or stress values for the predetermined health state, the statistical data being acquired in advance.

9. The analysis system according to claim 1,
wherein the personal information further comprises information regarding work in the organization,
wherein the processor is configured to extract the member of the adjustment target based on the information regarding the work in the organization, and
wherein the processor is configured to adjust the set of the fatigue and/or stress values by correcting the fatigue and/or stress value of the member of the adjustment target or by excluding the fatigue and/or stress value thereof from the analysis.

10. The analysis system according to claim 1,
wherein the at least one memory or storage device is further configured to store personal confidential information that is kept confidential to the organization,
wherein processor is configured to analyze the fatigue and/or stress state of the organization based on the stored personal information stored and the stored personal confidential information.

11. The analysis system according to claim 1, wherein the processor is configured to analyze the fatigue and/or stress state of the organization by classifying the fatigue and/or stress values into a plurality of categories depending on magnitudes of the fatigue and/or stress values.

12. The analysis system according to claim 11, further comprising a display configured to graphically display an analysis result of the fatigue and/or stress state of the organization.

13. The analysis system according to claim 1, wherein the information regarding the fatigue and/or the stress comprises biological information of the plurality of members.

14. The analysis system according to claim 3, wherein the information regarding the fatigue and/or the stress comprises biological information of the plurality of members.

15. An analysis method for analyzing a fatigue and/or stress state of an organization to which a plurality of members belongs, the analysis method comprising the steps of:
acquiring information regarding fatigue and/or stress of the plurality of members;
acquiring personal information of the plurality of members, the personal information comprising health information or information about life events;
calculating a fatigue and/or stress value for each of the plurality of members based on the information regarding the fatigue and/or the stress;
extracting the member of an adjustment target from among the plurality of members based on the personal information and a predetermined condition for extracting the member of the adjustment target, the predetermined condition comprising whether a predetermined health state included in the health information is good or whether a predetermined life event has occurred;
adjusting a set of the fatigue and/or stress values by correcting the fatigue and/or stress value of the member of the adjustment target or by excluding the fatigue and/or stress value thereof from an analysis on condition that the personal information takes the predetermined value; and
analyzing the fatigue and/or stress state of the organization based on an adjusted set of the fatigue and/or stress values.

16. A non-transitory computer-readable medium having instructions stored thereon that, when executed by a computer, cause the computer to perform the method according to claim 15.
